(19) United States
(12) Patent Application Publication
(10) Pub. No.: US 2009/0081170 A1
(43) Pub. Date: Mar. 26, 2009

(54) Cardiac progenitor cells

(76) Inventor: Paul Riley, London (GB)

Correspondence Address: KLAUBER & JACKSON, 411 HACKENSACK AVENUE, HACKENSACK, NJ, 07601, US

(21) Appl. No.: 12/283,741

(22) Filed: Sep. 15, 2008

Related U.S. Application Data
(60) Provisional application No. 60/993,618, filed on Sep. 13, 2007.

(51) Int. Cl. A61K 35/34; C12N 5/00; C12N 5/02; A61K 38/22; A61P 9/00; C12Q 1/02; A01K 67/027

(52) U.S. Cl. 424/93.7; 435/325; 435/378; 435/377; 435/29; 800/13; 514/12

(57) ABSTRACT

The present invention relates to the field of progenitor cells, and in particular to the field of cardiac progenitor cells. More particularly, the present invention pertains to the identification of a population of progenitor cells in the adult mammalian heart that is capable of giving rise to significant levels of de novo cardiomyocytes with the potential to replenish injured muscle post-infarction and/or promote neovascularisation to bring about complete cardiac regeneration. Accordingly, the present invention relates to methods for generating a population of mammalian post-natal epicardium derived cells (EPDCs), populations of EPDCs so generated, and methods of using same.

### CROSS REFERENCE TO RELATED APPLICATION

This application claims priority under 35 USC §119(e) from U.S. Provisional Application Ser. No. 60/993,618, filed Sep. 13, 2007, which application is herein specifically incorporated by reference in its entirety.

### TECHNICAL FIELD

This invention is in the field of progenitor cells, and in particular in the field of cardiac progenitor cells.

### BACKGROUND ART

Several publications and patent documents are referenced in this application in order to more fully describe the state of the art to which this invention pertains. The disclosure of each of these publications and documents is incorporated by reference herein.

For heart attack victims the prognosis for long term survival is poor. Necrotic myocardium, arising from acute myocardial infarction (MI), is replaced by non-contractile scar tissue/fibrosis [1], and spared cardiac muscle undergoes pathological hypertrophy to recover contractile force. This leads to pathological remodelling in the form of infarct expansion, thinning of the infarct wall and regional dilatation [2], the outcome of which is sub-optimal cardiac function, future MI events and the distinct possibility of fatal cardiac rupture and organ failure. Approaches to curing or mitigating effects of myocardial dysfunction have focused on replacement of damaged myocardium with healthy myocytes and the induction of new vessel formation to sustain both new and retained cardiac muscle.

A major shortcoming of current angiogenic therapy in response to myocardial ischaemia in humans is that the outcome may be limited to capillary growth without concomitant collateral support of arterioles [3].

Recent evidence suggests that a population of extracardiac or intracardiac stem cells may contribute to maintenance of the cardiomyocyte population, and thus cardiac muscle, under normal circumstances [4 to 6]. Although the stem cell population may maintain a delicate balance between cell death and cell renewal, it is insufficient for myocardial repair after acute coronary occlusion. Vascular regeneration includes adaptive vasculogenesis and arteriogenesis [7], and the supply of endothelial and smooth muscle vascular precursors required for this process has been attributed, in part, to the peripheral circulation and bone marrow [8,9].

To develop a method of curing or mitigating effects of myocardial dysfunction, significant effort has been invested in cell transplantation strategies with autologous bone marrow derived stem cells [reviewed in reference 10] and in the search for embryonic [11 to 13] or adult cardiac progenitor cells [14], which may replace damaged muscle cells and/or contribute to neovascularisation. Key to success of the latter is the identification of factors which may induce endogenous progenitor cells to initiate myocardial repair and collateral vessel growth.

Despite this work, only a single rare c-kit positive population of cardiac stem cells from the myocardium has thus far been identified with a limited and contentious capacity to contribute to cardiac repair [15, 16]. There is consequently a pressing requirement for the identification of a population of progenitor cells in the adult mammalian heart which can give rise to both significant levels of de novo cardiomyocytes with the potential to replenish injured muscle post-infarction and/or promote neovascularisation to bring about complete cardiac regeneration.

### DISCLOSURE OF THE INVENTION

The adult epicardium, unlike that of the embryonic epicardium, has come to be regarded as a quiescent lineage, incapable of migration or differentiation. As such, the adult epicardium has been viewed as incapable of giving rise to de novo cardiomyocytes or cells which are capable of neovascularisation, migration or differentiation. The inventors have, however, surprisingly identified the adult epicardium as a source of progenitor cells which, upon appropriate stimulation, can migrate and differentiate into endothelial and smooth muscle cells (vascular precursors). Unexpectedly, the population of cells identified by the inventors can also give rise directly to new cardiomyocytes and to fibroblasts.

In contrast to normal adult heart cell populations, the cell populations identified by the inventors show extensive outgrowth of cells which, like embryonic cultures, display a characteristic epithelial morphology and are positive for the epicardial-specific transcription factor, epicardin. The populations of cells of the invention appear to be reprogrammed to an embryonic fate and express embryonic genes such as Tbx18 [12] and Raldh2 [13].

This is the first example of a population of progenitor cells from the adult heart that are capable of differentiating into smooth muscle, endothelial cells cardiomyocytes and fibroblasts. This population of progenitor cells are referred to as post-natal epicardium-derived cells (EPDCs).

As used herein, the term “progenitor cells” refers to undifferentiated cells with the capacity for self-renewal, via a limited number of cell divisions, and differentiation.

The term “precursor cells” as used herein refers to partially committed cells that divide and give rise to certain types of cells, but are not capable of developing into all the cell types of a tissue.

### EPDCs

In the first aspect of the invention, the invention provides a population of post-natal epicardium derived cells (EPDCs), wherein at least 50% of said EPDCs express at least one embryonic gene.

By “post-natal” is meant that the population of cells is derived from the epicardium of the mammalian heart after birth. Preferably, the cells are derived from the epicardium of an adult mammal. Preferably, the cells are derived from rodent or primate epicardium, preferably human epicardium.

By “express” is meant that the gene produces an mRNA or protein product at detectable levels within the cell. Preferably, the EPDCs express at least one embryonic gene at levels that are similar to the levels of expression detected in embryonic cells. Gene expression can be detected by standard methods known in the art. Gene expression can be measured by detecting mRNA using northern Blotting, quantitative real time PCR (qRT-PCR), RT-PCR or any other method known in the art. Gene expression can be measured by detecting the protein encoded by the gene using FACs, western Blotting, immunostaining or any other method known in the art.

The results presented herein demonstrate the existence of a post-natal population of epicardial cells that express Tbx18 [17] and Raldh2 [18], genes that are normally expressed during embryonic development. The ability of the EPDCs of the invention to express embryonic genes is indicative that these cells in the adult mammalian epicardium have been reprogrammed to an embryonic fate.

Preferably, at least 50% of the EPDCs express at least one of the embryonic genes Tbx18 and Raldh2. Preferably, at least 50% of the EPDCs express both Tbx18 and Raldh2. In a preferred embodiment, 60%, 70%, 80%, 90%, 95%, 99% or more of the EPDCs express both of the embryonic genes Tbx18 and Raldh2.

Although the inventors do not wish to be bound by theory, it appears likely that the EPDCs of the invention may express further embryonic genes and the invention thus encompasses EPDCs expressing further embryonic genes, for example Gata5 [19].

The EPDCs of the invention may also express epicardial specific markers. In one embodiment, at least 50% of the EPDCs of the invention express the epicardial-specific transcription factor epicardin [20]. Preferably, 60%, 70%, 80%, 90%, 95%, 99% or more of the EPDCs of the invention express epicardin.

Although the inventors do not wish to be bound by theory, it appears likely that the EPDCs of the invention may express further epicardial specific markers and the invention thus encompasses EPDCs expressing further epicardial specific markers, for example Gata5, WT-1 and cytokeratin [21, 22].

The expression of these embryonic markers and epicardial markers may be measured by any method known in the art, for example Northern Blotting, quantitative real time PCR (qRT-PCR), RT-PCR and/or western blotting. In particular, immunostaining using antibodies against these markers may be employed to identify the proportion of cells expressing the markers.

EPDCs possess certain properties that are usually observed in embryonic epicardial cells, but not in post-natal epicardial cells.

For example, the EPDCs of this aspect of the invention may display epithelial morphology characteristic of embryonic cells. By “epithelial morphology” is meant that the EPDCs form flat monolayers characteristic of epithelial cells. In one embodiment, at least 50% of the EPDCs of the invention display epithelial morphology. Preferably, 60%, 70%, 80%, 90%, 95%, 99% or more of the EPDCs of the invention display epithelial morphology.

The EPDCs are preferably also proliferative. By “proliferative” is meant that the cells are capable of expansion by cell division. Preferably, the EPDCs of the invention express markers associated with proliferation such as Ki67 [23] and/or phospho-histone H3 [24]. Therefore, in an embodiment of the first aspect, at least 50% of the EPDCs express Ki67 and/or phospho-histone H3. Preferably, at least 60%, 70%, 80%, 90%, 95%, 99% or more of the EPDCs express Ki67 and/or phospho-histone H3.

The EPDCs are preferably capable of migration away from the epicardium both in cell culture in vitro and in the epicardium in vivo. The ability of EPDCs to migrate away from the epicardium in vitro may be assessed by visual inspection of cells in culture. In vivo, cell migration may be measured by lineage tracing, a process by which EPDCs are labelled (e.g. with a fluorescent marker) and the migration of these cells in the heart can be traced directly [25]. Therefore, in an embodiment of the first aspect, at least 50% of the EPDCs are capable of migration. Preferably, at least 60%, 70%, 80%, 90%, 95%, 99% or more of the EPDCs are capable of migration.

The EPDCs of the first aspect of the invention are undifferentiated cells that have not yet developed into one or more specialised cell types. The EPDCs of the invention are also multipotent. By “multipotent” is meant that the EPDCs can differentiate into several other cell types, but those types are limited in number. The EPDCs of the invention are multipotent cells which can differentiate into vascular precursor cells, cardiomyocytes and fibroblasts. The vascular precursor cells derived from EPDCs are also multipotent and can further differentiate into smooth muscle cells and endothelial cells.

In an embodiment of the first aspect, at least 50% of the EPDCs are capable of differentiation into vascular precursor cells, cardiomyocytes and/or fibroblasts. Preferably, 60%, 70%, 80%, 90%, 95%, 99% or more of the EPDCs are capable of differentiation into vascular precursor cells, cardiomyocytes and/or fibroblasts. Preferably, a population of EPDCs according to the invention is capable of differentiation into vascular precursor cells, cardiomyocytes and fibroblasts. As noted above, the EPDCs of the invention are the first population of cells isolated from adult mammalian epicardium that are capable of differentiating into all three types of cardiac cells.

Preferably, the EPDCs of the invention are capable of differentiation into vascular precursor cells, cardiomyocytes and/or fibroblasts when cultured in the presence of Thymosin β4 (Tβ4) or a functional equivalent thereof. The EPDCs of the invention are capable of differentiation into vascular precursor cells, cardiomyocytes and/or fibroblasts when cultured using the protocols described herein. In particular, the EPDCs of the invention are capable of differentiation into vascular precursor cells, cardiomyocytes and/or fibroblasts when cultured with 10 to 500 ng/ml Tβ4 or a functional equivalent thereof, more preferably with 50 to 250 ng/ml Tβ4 or a functional equivalent thereof, even more preferably with 100 ng/ml Tβ4 or a functional equivalent thereof.

Vascular endothelial and smooth muscle cells and cardiomyocytes derived from the EPDCs of the invention by differentiation are themselves a further aspect of the invention. Such cells are referred to herein as “cells derived from EPDCs” or “EPDC-derived cells”.

In a further embodiment of the first aspect, EPDC-derived vascular precursor cells are capable of differentiating into endothelial cells and smooth muscle cells. Both smooth muscle and endothelial cells are derived from vascular precursor cells and are required for neovascularisation.

“Neovascularisation” is the formation of new, functional blood vessels. As used herein, “neovascularisation” includes: vasculogenesis, the de novo formation of vessels; angiogenesis, the growth of new blood vessels from pre-existing vessels; and arteriogenesis, an increase in the diameter of existing vessels.

Preferably, at least 50% of EPDC-derived vascular precursor cells can give rise to endothelial cells and smooth muscle cells. Even more preferably, 60%, 70%, 80%, 90%, 95%, 99% or more of the EPDC-derived vascular precursor cells can give rise to endothelial cells and smooth muscle cells

Smooth muscle is a type of non-striated muscle, found in the vasculature and other organs. Smooth muscle cells make up the majority of the wall of blood vessels. Smooth muscle cells express one or more of the markers SMαA and SM22α.

The walls of blood vessels also contain endothelial cells. Endothelial cells express markers including Flk1, Tie2, PECAM, and/or VEGF.

In one embodiment of the first aspect, at least 50% of the endothelial cells derived from EPDCs express at least one of the markers Flk1, Tie2, PECAM, and/or VEGF. Preferably, at least 60%, 70%, 80%, 90%, 95%, 99% or more of the endothelial cells derived from EPDCs express at least one of the markers Flk1, Tie2, PECAM, and/or VEGF

In another embodiment of the first aspect, at least 50% of the smooth muscle cells derived from EPDCs express one or more of the markers SMαA and SM22α. Preferably, at least 60%, 70%, 80%, 90%, 95%, 99% or more of the smooth muscle cells derived from EPDCs express the one or more of the markers SMαA and SM22α.

Smooth muscle cell markers (such as SMαA and SM22α) and endothelial cell markers (such as Flk1, Tie2, PECAM, and/or VEGF) can be detected by any methods known in the art, including RT-PCR, quantitative real time PCR (qRT-PCR), western blotting and immunostaining as described herein.

EPDCs can also differentiate into cardiomyocytes and fibroblasts.

Cardiomyocytes are the cells that make up the heart muscle. Cardiomyocyte precursor cells express at least one of the markers Isl-1, Nkx2.5 and/or Gata 4. Terminally differentiated cardiomyocytes express at least one of the markers sarcomeric, α-actin, α-myosin heavy chain, cardiac myosin binding protein c and/or cardiac triponin-T. These markers can be detected by RT-PCR, quantitative real time PCR (qRT-PCR), western blotting and immunostaining as described herein.

Therefore, in a further embodiment of the first aspect, at least 50% of the cardiomyocytes derived from EPDCs express at least one of the markers Isl-1, Nkx2.5, Gata 4, sarcomeric, α-actin, α-myosin heavy chain, cardiac myosin binding protein c and/or cardiac triponin-T. Preferably, 60%, 70%, 80%, 90%, 95%, 99% or more of the cardiomyocytes derived from EPDCs express at least one of the markers Isl-1, Nkx2.5, Gata 4, sarcomeric, α-actin, α-myosin heavy chain, cardiac myosin binding protein c and/or cardiac triponin-T.

Fibroblasts are a type of cell that synthesizes and maintains the extracellular matrix of animal tissues. Fibroblasts are also involved in wound repair and scar formation. Fibroblasts express procollagen type I. Procollagen type I can be detected by RT-PCR, western blotting and immunostaining as described herein.

Therefore, in one embodiment, at least 50% of the fibroblasts derived from EPDCs express procollagen type I. Preferably, at least 60%, 70%, 80%, 90%, 95%, 99% or more of the fibroblasts derived from EPDCs express procollagen type I.

In a further embodiment, the EPDCs and EPDC-derived vascular precursor cells of the invention are capable of neovascularisation in vivo and in vitro. The ability of the EPDCs and EPDC-derived cells of the invention to promote neovascularisation may be measured by detecting markers for endothelial cells, such as Flk1, Tie2, PECAM, and/or VEGF, and markers for smooth muscle cells, such as SMαA, by immunostaining. Immunostaining can be carried out in vitro or in vivo by any of the methods known in the art, for example as described in reference 26. In vivo, neovascularisation can be determined visually by tracking the formation of new vessels using, for example, MRI with or without arterial spin labelling [27]. Neovascularisation can also be determined visually in vitro by the formation of vessel-like structures. In particular, the formation of vessel-like structures can be detected when EPDCs are cultured in matrigel or on other scaffold-like structures.

The populations of EPDCs of the first aspect of the invention can be cultured in vitro or can be induced in vivo. In in vitro culture, the population of EPDCs can be expanded to any size, and may typically contain 106 to 1010 cells, or more. For example, an in vitro population of EPDCs may contain 10⁶, 10⁷, 10⁸, 10⁹, 10¹⁰ or more cells. In vivo, the population of EPDCs may also contain 10⁶ cells or more. For example, an in vivo population of EPDCs may contain 10⁶, 10⁷, 10⁸, 10⁹, 10¹⁰ or more cells.

### Methods of Obtaining EPDCs

In a second aspect, the invention also provides methods of obtaining a population of isolated post-natal epicardial cells according to the first aspect of the invention comprising the steps of culturing heart tissue explants in culture medium comprising thymosin P4 (Tβ4) or a functional equivalent thereof for sufficient time to permit EPDC outgrowth.

Tβ4 is expressed at high levels in the embryonic heart [28]. However, expression of Tβ4 drops to almost undetectable levels in the post-natal and adult heart. In particular, Tβ4 expression in the post-natal epicardium is too low to permit activation of EPDCs. However, the present inventors have surprisingly found that when a post-natal heart tissue or epicardial explant is exposed to about 10 to 500 ng/ml Tβ4 or a functional equivalent thereof, it is possible to obtain a population of EPDCs according to the first aspect of the invention.

By “EPDC outgrowth” is meant EPDC proliferation and migration away from the initial explant. Outgrowth can be monitored visually, by inspecting the culture for spread of EPDCs away from the explants.

The method may also comprise the further steps of:
a) washing the cells with DPBS, and
b) adding fresh culture medium containing Tβ4 or a functional equivalent thereof.

The heart tissue explants employed in the method of the second aspect of the invention may be of any size that is suitable to permit tissue adhesion to a tissue culture dish and is sufficiently large to provide a sustainable population of EPDCs. Preferably, the explants are from about 0.5 to about 5 mm³, more preferably about 0.75 to about 3 mm³, even more preferably about 1 mm³.

The explant is preferably treated with about 10 to 500 ng/ml Tβ4 or a functional equivalent thereof, more preferably with 50 to 250 ng/ml Tβ4 or a functional equivalent thereof, even more preferably with 100 ng/ml Tβ4 or a functional equivalent thereof.

Tβ4 is a G-actin monomer binding protein implicated in reorganization of the actin cytoskeleton, a process fundamentally required for cell migration. Ectopic administration of Tβ4 in a mouse model of MI has shown to reduce scarring and improve cardiac function via Akt-induced cardiomyocyte survival [28]. However, the results presented herein show for the first time that culture of cells with Tβ4 results in re-programming of a population of cells within the adult mammalian epicardium to express embryonic markers, and that continued culture of these EPDCs with Tβ4 induces the EPDCs to differentiate into vascular precursors, cardiomyocytes and fibroblasts. Continued culture of the vascular precursors with Tβ4 results in differentiation into endothelial and smooth muscle cells.

The murine Tβ4 polypeptide sequence has been given accession number GI:10946578 in the Entrez protein database and the mRNA sequence is given in GI:86476080. The human Tβ4 polypeptide sequence has been given accession number GI:11056061 in the Entrez protein database and the mRNA sequence is given in GI:34328943.

The term “functional equivalent” is used to describe homologues and fragments of Tβ4 which retain the ability to promote outgrowth of EPDCs of the first aspect of the invention from heart tissue explant cultures. Preferably, functional equivalents of Tβ4 retain the ability to promote the formation of EPDCs having all of the characteristics discussed above in connection with the first aspect of the invention.

Methods for the identification of homologues of Tβ4 are known in the art. Preferably, proteins that are homologues have a degree of sequence identity with Tβ4 of greater than 70%, 80%, 90%, 95%, 98% or 99%, respectively. Percentage identity, as referred to herein, is as determined using BLAST version 2.1.3 using the default parameters specified by the NCBI (the National Center for Biotechnology Information; http://www.ncbi.nlm.nih.gov/) [Blosum 62 matrix; gap open penalty=11 and gap extension penalty=1].

Homologues of Tβ4 include mutants containing amino acid substitutions, insertions or deletions from the wild type sequence, provided that the ability to promote outgrowth of EPDCs of the first aspect of the invention from heart tissue explant cultures is retained. Mutants thus include proteins containing conservative amino acid substitutions that do not affect the function or activity of the protein in an adverse manner. Fragments of Tβ4 and of homologues of Tβ4 protein are also provided by the invention. Preferred fragments include fragments comprising or consisting of the G-actin binding domain of Tβ4 and fragments comprising or consisting of the N-terminal tetrapeptide N-acetyl-seryl-aspartyllysyl-proline (AcSDKP). Fragments with improved activity in promoting EPDC outgrowth may, of course, be rationally designed by the systematic mutation or fragmentation of the wild type sequence followed by appropriate activity assays.

The term “functional equivalent” also refers to molecules that are structurally similar to Tβ4 or that contain similar or identical tertiary structure, particularly in the environment of the active site or active sites of Tβ4.

The culture medium in which the method of the second aspect of the invention is carried out may be a standard culture medium to which Tβ4 has been added. For example, the method of the second aspect of the invention may be carried out in DMEM containing GlutaMaxI and about 4.5 g/L glucose, supplemented with about 15% FBS; about 100 units/ml penicillin; and about 100 μg/ml streptomycin.

The explants may be cultured for 12 hours, 24 hours, 36 hours, 72 hours or longer, to permit EPDC outgrowth. The washing step may take place after 12 hours, 24 hours, 36 hours, 72 hours or longer. The step of adding fresh medium containing Tβ4 may be followed by a further step of culturing the EPDCs for 12 hours, 24 hours, 36 hours, 72 hours or longer.

The explants may be derived from any region of outer heart tissue containing the overlying epicardium. Preferably, the explants are specifically derived from the epicardium.

The invention also provides a population of EPDCs obtained or obtainable by any of the methods described herein. The EPDCs obtained by the methods described above are useful in screening assays and in methods of treatment as described herein.

Methods of growing tissue from EPDCs

The EPDCs described herein may be used to grow tissues in vitro. In particular, heart muscle and vascular tissue can be grown in culture.

According to a further aspect of the invention, there is thus provided a method of growing vascular tissue in vitro comprising culturing EPDCs of the first aspect of the invention in a culture medium comprising Tβ4 or a functional equivalent thereof. Preferably, the EPDCs are obtained according to the method of the second aspect of the invention and continue to be cultured in Tβ4 until the EPDCs differentiate into vascular tissue. Preferably, the method comprises supplying the culture with a 3D matrix, such as a matrigel or a scaffold, to promote the formation of new blood vessels [29].

The invention also provides a method of growing myocardial tissue in vitro comprising culturing EPDCs of the first aspect of the invention in a culture medium comprising Tβ4 or a functional equivalent thereof. Preferably, the EPDCs are obtained according to the method of the second aspect of the invention and continue to be cultured in Tβ4 until the EPDCs differentiate into myocardial tissue. Such a method may be carried out in a tissue culture dish.

The culture medium may contain additional growth factors that promote the formation of heart muscle and vascular tissue, such as VEGF and FGFs.

### Animal Models

In many research and medical applications, animal models are useful tools. The inventors have determined that Tβ4 plays a key role in development of the heart. This is the first time that a single factor has been shown to be involved in the development of all types of cells required for cardiac development and regeneration.

The present invention therefore provides transgenic non-human animals, and tissues or cells derived therefrom, wherein Tβ4 expression in the heart of the transgenic animal is altered. In one aspect, the Tβ4 expression is altered only in the epicardium. Preferably, Tβ4 expression in the heart is reduced or eliminated.

In one aspect, the non-human transgenic animal is a mouse, a rat, a pig or a primate.

In a further aspect, the invention provides EPDCs derived from the transgenic, non-human animal.

Tβ4 expression may be reduced or eliminated using any method know in the art, for example by the expression of a nucleic acid or nucleic acid fragment that is antisense to the Tβ4 gene. Gene silencing may also be used, for example using RNAi and/or siRNA. In a preferred embodiment, the Tβ4 expression is conditionally knocked-down in the heart using RNAi.

In a further preferred embodiment, the transgenic non-human animal expresses the construct Tβ4shRNAflox described herein and either one of the constructs Nkx2-5CreKI, which directs Cre expression throughout the majority of cardiomyocytes [30, 31] or MLC2vCreKI, which directs Cre expression specifically to ventricular cardiomyocytes [32], both also described herein.

These transgenic non-human animals will be useful as research tools to establish the role of Tβ4 in cardiac function by comparing cardiac function in normal mice with cardiac function Tβ4-null mice. For example, a comprehensive assessment of Tβ4-null hearts for any gross defects over the time course of postnatal growth may be carried out and histological sections may be examined to discern any gross abnormalities in cardiac structures, alteration in chamber size or shape or wall thickness which may be measured by MRI. Extent of fibrosis may be assessed by collagen staining with Masson's trichrome. Measurements of stroke volume, ejection fraction and wall thickness [33 to 35] may be recorded and myocardial wall motion may be assessed by tagging strain analysis [36]. Fibrosis and scarring may be visualised at the final imaging time point using delayed hyper-enhancement of the MRI contrast agent gadolinium [43, 37].

The transgenic non-human animals may also be used to study the extent to which Tβ4 is required for maintaining cardiomyocyte ultrastructure, function and viability. Neonatal and adult cardiomyocytes from Tβ4-null and control animals may be examined for cytoskeletal disruption resulting from Tβ4 loss by staining of actin filaments with Alexa488-phalloidin and confocal microscopy. In particular, the transgenic non-human animals of the invention may be used to assess the effect of loss of Tβ4 on the incidence of stress fibre formation and defects in muscle ultrastructure (sarcomeric disorganisation). Levels of apoptotic cell death in the transgenic non-human animals may assayed by TUNEL staining (DeadEnd Colorimetric System, Promega). The actin cytoskeleton plays a central role in modulating the electrical activity, through ion channels and exchangers, and the mechanical (contractile) properties of the heart. Loss of Tβ4, and its effect on the cytoskeleton, may therefore directly influence the electrical activity of cardiomyocytes. The ion channel profile of Tβ4-null cardiomyocytes from the transgenic non-human animals of the invention may therefore be assessed by patch-clamp analysis and the ability of Tβ4-to restore the ion channel activity of these cells may be assessed.

The transgenic non-human animals may also be used to assess the condition of the coronary vasculature. For example, capillary vessel density and lumen area may be measured in the epicardial, endocardial and midmyocardial portions of the left ventricle by morphometric analysis after immunostaining of capillaries for PECAM-1 and CD31 and arteries for anti-SMαA. The degree of branching from the main coronary arteries may be assessed by immunoconfocal reconstruction of arteriolar trees labelled for SMαA in thick (100 μm) longitudinal sections of the left ventricle [38]. The functional capacity of the coronary vasculature may be assessed by MRI under resting conditions and following recovery from ischemic injury. A MRI technique known as arterial spin labelling [43] will be used to determine cardiac perfusion. Magnetic resonance angiography will be performed to assess major vessels and coronary arteries [39].

The transgenic animals of the invention will also be useful in the screening assays described below.

### Screening Assays

The identification of a population of EPDCs capable of differentiating into vascular precursor cells, cardiomyocytes and fibroblasts enables screening to be conducted for compounds that promote the formation of one or more of these cell types. Such compounds have potential therapeutic benefits in the treatment of diseases and disorders of the heart such as inflammation and MI. In addition, the population of cells is useful in screening methods for use in research and drug development.

### Systems for Carrying Out Screening Assays

Screening methods of the invention are carried out using EPDC populations and EPDC-derived populations described above. Preferably the screening method is carried out in a human EPDC population.

The screening methods may be carried out in cell cultures in vitro or animal models in vivo. In particular, screening methods may be carried out in the non-human transgenic animals having depleted or deleted Tβ4 described above. Screening in a loss of Tβ4 function background will enable any drug candidate's effects to be specifically determined in isolation and without potential background effects of endogenous Tβ4.

### Screening Assays

The invention therefore provides a method of screening for a compound that promotes vascular precursor cell formation, comprising the steps of:
exposing a population of EPDCs according to the first aspect of the invention to a candidate compound, and
comparing vascular precursor cell formation in the presence and absence of the candidate compound.

Vascular precursor cell formation can be measured in vitro or in vivo by detection of markers known to be associated with smooth muscle cell or endothelial cell formation by RT-PCR, western blotting and/or immunostaining. Markers that may be detected include the markers discussed above, namely Flk1, Tie2, PECAM, VEGF, and/or SMαA. Fluorescent activated cells sorting (FACS) analysis can also be used to detect the number of cells that express any one of these markers. In addition, the extent of enhanced perfusion of the hearts brought about by new vasculature in vivo can be determined in animal models by cardiac injections of rhodamine or TRITC-conjugated dextran.

The invention also provides a method of screening for a compound that promotes cardiomyocyte formation, comprising the steps of:
exposing a population of cells according to the invention to a candidate compound, and
comparing cardiomyocyte formation in the presence and absence of the candidate compound.

Cardiomyocyte formation can be measured in vitro or in vivo by detection of cardiomyocyte markers, for example by RT-PCR, western blotting and/or immunostaining to detect the markers Isl-1, Nkx2.5, Gata 4, sarcomeric, α-actin, α-myosin heavy chain, cardiac myosin binding protein c and/or cardiac triponin-T. Fluorescent activated cells sorting (FACS) [40] analysis can be used to detect the number of cells that express any one of these markers. The formation of cardiomyocytes can also be detected in vitro by identifying cells that beat [41]. Patch clamping can also be used to identify cardiomyocytes and attribute functional contraction via the recording of action potentials [42].

In vivo, cardiomyocyte formation can be measured in animal models where the new cardiomyocytes can be genetically traced and their functional integration assessed with resident cardiomyocytes via expression of gap junction proteins such as connexin 43 (Cx-43).

The invention further provides a method of screening for a compound that promotes neovascularisation, comprising the steps of:
exposing a population of cells according to the invention to a candidate compound, and
comparing neovascularisation in the presence and absence of the candidate compound.

In vitro, neovascularisation can be measured by any method known in the art, for example by RT-PCR, western blotting and/or immunostaining to detect the markers Flk1, Tie2, PECAM, VEGF, and/or SMαA. FACS analysis can be used to detect the number of cells that express any one of these markers. Formation of vessel-like structures in culture, for example in matrigel, can also be used to measure neovascularisation.

In vivo, neovascularisation can be measured by detecting the formation of new vessels. New vessel formation can be measured by any method known in the art, for example MRI with or without arterial spin labelling [43].

The development of vessel-like structures may be monitored by monitoring the lengths of projections and degree of branching in vivo. Immunofluorescence and confocal microscopy may be used to identify endothelial (Flk-1, Tie 2, PECAM) and smooth muscle (SMαA, SM22α) cells within the vessels and the temporal expression of potential effectors and markers (epicardin, PECAM, Flt1, Flk1, bFGF, VEGF, SM22α) may be assayed by RT-PCR or western analysis over the time course of vessel outgrowth.

Animal models may also be used to assess the ability of the compound to induce neovascularisation in vivo. Preferably, a gain of function model may be used to assess the ability of the compound to induce neovascularisation. For example, a gain of function mouse model may be developed from crosses between two transgenic strains: Gata5Cre (epicardial specific [44]) and R26R-EYFP (contain a targeted insertion of EYFP into the ROSA26 locus [45]). The resulting mice will have EYFP positive EPDCs and EPDC-derived progeny, such as endothelial and smooth muscle cells, which should persist into adulthood, enabling epicardial contribution to neovascularisation following administration of the compound to be tracked directly.

The invention further provides a method of screening for a compound that promotes fibroblast formation, comprising the steps of:
exposing a population of cells according to the invention to a candidate compound, and
comparing fibroblast formation in the presence and absence of the candidate compound.

Fibroblast formation can be measured by any method known in the art, for example by RT-PCR, western blotting and/or immunostaining to detect the marker procollagen al. Fluorescent activated cells sorting (FACS) analysis can be used to detect the number of cells that express any one of these markers.

It is possible that EPDC expansion and differentiation may alter in the presence of an injury response. The screening methods of the invention may therefore be carried out in an MI animal model, such as the Gata5Cre/R26R-EYFP mouse model, in which MI is recreated by ligation of the left anterior descending coronary artery, or Cx40-EGFP mice which have an EGFP positive coronary vasculature and conduction system [46].

Compounds can be screened for activity in one or more of the assays described above. The assays can also be used to identify compounds that do not promote one or more of vascular precursor cell, cardiomyocyte, fibroblast formation and/or neovascularisation.

In some instances, it is preferable for the compound to able to promote only one of vascular precursor cell, cardiomyocyte, fibroblast formation and/or neovascularisation. For instance, when a selective effect on vascular regeneration is desirable, compounds that are active only in promoting vascular precursor cell formation will be selected. For example, when coronary occlusion in present in the absence of MI (early onset of ischaemic heart disease), vascular cells would be preferable over cardiomyocytes. In other cases, it will be desirable to identify compounds that are active in a combination of assays, for example in promoting vascular precursor cell formation and cardiomyocyte formation, vascular precursor cell formation and fibroblast formation, cardiomyocyte formation and fibroblast formation, or vascular precursor cell, cardiomyocyte and fibroblast formation. In some cases, it may be desirable to identify compounds that promote neovascularisation in combination with the formation of any of vascular precursor cell, cardiomyocytes and fibroblasts, alone or in any combination. In some cases, it may be desirable to identify compounds that promote vascular precursor or cardiomyocyte formation without promoting fibroblast formation, for example to avoid fibrosis.

### REFERENCE STANDARDS

A reference standard (e.g. a control), is typically needed in order to detect whether the vascular precursor cell formation, cardiomyocyte formation and/or neovascularisation is increased. For example, in order to detect whether a candidate compound has the desired effect, the vascular precursor cell formation, cardiomyocyte formation and/or neovascularisation in the presence of a candidate compound may be compared with the vascular precursor cell formation, cardiomyocyte formation, and/or neovascularisation in the absence of a candidate compound.

The reference may have been determined before performing the method of the invention, or may be determined during (e.g. in parallel) or after the method has been performed. It may be an absolute standard derived from previous work.

### Candidate Compounds

Typical candidate compounds for use in all the screening methods of the invention include, but are not restricted to, peptides, peptoids, lipids, metals, small organic molecules, RNA aptamers, antibodies (as used herein, the term “antibody” refers to intact molecules as well as to fragments thereof, such as Fab, F(ab′)2 and Fv, which are capable of binding to the antigenic determinant in question) or antibody derivatives (e.g. antigen-binding fragments, single chain antibodies including scFvs, etc.), and combinations or derivatives thereof.

Peptides include functional equivalents of Tβ4 such as those described above in connection with the method of the second aspect of the invention. Additional candidate compounds may be compounds that act on the Tβ4 receptor or on other compounds to which Tβ4 binds. In particular, candidate compounds may include molecules in the Akt/integrin signalling pathways [28] and angiogenic factors including VEGF and FGFs. Candidate compounds may also include compounds that up-regulate the level or activity of Tβ4.

Small organic molecules have a molecular weight of about more than 50 and less than about 2,500 daltons, and most preferably between about 300 and about 800 daltons. Candidate compounds may be derived from large libraries of synthetic or natural compounds. For instance, synthetic compound libraries are commercially available from MayBridge Chemical Co. (Revillet, Cornwall, UK) or Aldrich (Milwaukee, Wis.). Alternatively, libraries of natural compounds in the form of bacterial, fungal, plant and animal extracts may be used. Additionally, candidate compounds may be synthetically produced using combinatorial chemistry either as individual compounds or as mixtures.

In some instances, it may be desirable to conduct a preliminary screening step to reduce the number of candidate compounds used in the methods of the invention. For example, a preliminary assay may be carried out to identify candidate compounds that bind to Tβ4, functional equivalents of Tβ4, or to receptors of Tβ4 that may then be used in the screening methods of the invention. Alternatively, preliminary assays may be carried out to identify candidate compounds that bind to up-regulate the level of expression of Tβ4 or functional equivalents of Tβ4.

### In Vivo Confirmation of Function of Compounds Identified

Once a compound has been identified, it may be desirable to perform further experiments to confirm the in vivo function of the compound.

The invention therefore provides a method of assessing the in vivo effect of a compound obtained or obtainable by any of the methods described above comprising administering the compound to a test animal and assessing the effect on the test animal. The step of assessing the effect on the test animal may comprise the step of assessing its effect of vascular precursor cell formation, cardiomyocyte formation, fibroblast formation and/or neovascularisation.

Tests in non-human animals, for example non-human rodents or non-human primates may be used. The non-human transgenic animals described herein may also be used.

Compounds Identified by Screening Methods and Methods of Treatment Employing these Compounds

The invention provides a compound that promotes the vascular precursor cell formation, cardiomyocyte formation, fibroblast formation and/or neovascularisation.

Once identified, these compounds can be used in methods to promote vascular precursor cell formation, cardiomyocyte formation, fibroblast formation and/or neovascularisation.

Once a compound has been identified using one of the methods of the invention, it may be necessary to conduct further work on its pharmaceutical properties. For example, it may be necessary to alter the compound to improve its pharmacokinetic properties or bioavailability. The invention extends to any compounds obtained or obtainable by the methods of the invention which have been altered to improve their pharmacokinetic properties.

The compounds identified by the screening methods of the invention may be used in the treatment of cardiac disorders. The invention thus extends to methods of treating cardiac disorders including MI, cardiac inflammation and cardiac degeneration comprising administering a compound identified by a screening method of the invention to a patient in need thereof.

### EPDCs and Myocardial Regeneration

An important goal in treating diseases that affect/injure the heart, including ischaemic heart disease resulting in MI, is the regeneration of the myocardium. In general, the adult mammalian heart can not regenerate.

The ability of populations of cells of the invention to promote coronary vascularization in the adult enhances cardiomyocyte survival and contributes significantly towards cardiac regeneration.

The EPDCs and EPDC-derived cells of the invention are capable of giving rise to cells with the potential to replenish injured heart muscle and vasculature, in particular post-injury, post-infection and/or post-MI. Cardiac regeneration, which includes both myocardial and vascular regeneration, is required after any kind of cardiac injury, of which acute MI is the most common. Bacterial, viral and or fungal infection can also lead to cardiac inflammation and injury and as such therapeutics to promote cardiac regeneration would also be beneficial in these cases.

Effective myocardial regeneration requires new blood vessel formation and new cardiac muscle formation. The EPDCs of the invention can differentiate into all the cell types required for myocardial regeneration.

The invention thus provides a population of EPDCs or EPDC-derived cells according to the first aspect of the invention for use in therapy and, in particular, for use in the treatment of cardiac disease. The invention also provides a method of treating a cardiac disease in a patient in need thereof comprising administering to said patient a composition comprising a population of EPDCs or EPDC-derived cells according to the first aspect of the invention. The cardiac diseases that may be treated using the EPDCs and EPDC-derived cells according to the invention include MI and cardiac inflammation.

The invention also provides a population of EPDCs or EPDC-derived cells according to the invention for use in myocardial regeneration. The invention also provides a method of promoting myocardial regeneration in a patient in need thereof comprising administering a population of EPDCs or EPDC-derived cells according to the invention.

The EPDCs and/or EPDC-derived may be administered in combination with Tβ4 or a functional equivalent thereof. Preferably, the EPDCs and/or EPDC-derived cells are autologous. Autologous EPDCs and/or EPDC-derived cells may be obtained from patient cells isolated by biopsy and expanded in culture using the methods described herein. The invention thus provides a method of treating MI or cardiac inflammation, or promoting myocardial regeneration, comprising the administration of a combination of a population of EPDCs according to the first aspect of the invention and Tβ4 or a functional equivalent thereof. The invention also provides a combination of a population of cells and Tβ4 for use in therapy, and for use in treating MI or cardiac inflammation or promoting myocardial regeneration.

Preferably, the EPDCs and EPDC-derived cells employed in the therapeutic methods and uses of this aspect of the invention are autologous to the patient being treated to avoid rejection.

In the methods of treatment described herein, the population of cells may be delivered directly or in a biocompatible scaffold or matrix. Suitable biocompatible scaffolds and matrices are known in the art. Where the EPDCs are administered directly, they may be injected directly to the site of cardiac damage using catheter based approaches and in parallel with percutaneous reperfusion.

### Cardiac Inflammation

Cardiac regeneration is intricately linked to a complex inflammatory response that must be precisely regulated to ensure proper repair and optimal cardiac outcome. Persistence of the acute inflammatory response immediately post-MI is known to extend myocardial injury (reviewed in reference 47), however, moderate inflammation is almost certainly beneficial to repair given the requirement to both remove dead or dying cardiomyocytes post injury and resolve the infarct by scar formation [47].

The results presented herein show that Tβ4 modulates the acute inflammatory response to injury in the heart via a direct effect on the NFkB pathway thus tipping the balance from fibrosis/scarring in favour of regeneration.

The invention therefore provides Tβ4 for use in treating cardiac inflammation.

The invention also provides a method of treating inflammation in the heart comprising administering Tβ4. Inflammation may be caused by MI or any other source, for example infection of the heart by bacterial, viral or fungal pathogen.

### General

The term “comprising” encompasses “including” as well as “consisting” e.g. a composition “comprising” X may consist exclusively of X or may include something additional e.g. X+Y.

The word “substantially” does not exclude “completely” e.g. a composition which is “substantially free” from Y may be completely free from Y. Where necessary, the word “substantially” may be omitted from the definition of the invention.

The term “about” in relation to a numerical value x means, for example, x±10%.

Unless specifically stated, a process comprising a step of mixing two or more components does not require any specific order of mixing. Thus components can be mixed in any order. Where there are three components then two components can be combined with each other, and then the combination may be combined with the third component, etc.

### BRIEF DESCRIPTION OF THE DRAWINGS

FIG. 1 Frontal sections through the ventricular myocardium (my) of Tβ4shNk embryos at E14.5, stained with haematoxylin and eosin to visualize epicardial nodules, which represent aberrant coronary vessels; black arrowheads (a, b). SMαA-positive cells surround the cannular epicardial nodules (c). Tie2 immunofluorescence identifies endothelial cells in the control myocardium; white arrowheads (d). Tie2-positive cells are almost absent from Tβ4shNk myocardium (e) but many appear trapped in epicardial nodules as indicated by white arrowhead (f). Coronary vessels invading the control myocardium are surrounded by SMαA-positive cells; white arrowheads (g). Tβ4shNk myocardium contains very few vessels and is almost negative for SMαA (h). SMαA-positive cells appear trapped in the epicardium (ep) and compact layer in Tβ4shNk embryos; white arrowheads (i). Whole mount views (j, k) and frontal sections (l, m) of the developing aorta (ao) and pulmonary artery (pa) of control and Tβ4shNk embryos at E14.5 show weak vessels and absence of right subclavian artery (rsc; compare black arrowheads indicating position of rsc artery in j compared to k). Absence of SMαA-positive cells in the vessel walls of Tβ4shNk (o) embryos and a partial loss in Tβ4shMlc embryos (p), compared with control (n). ep, epicardium; my, myocardium.

FIG. 2. Tβ4 knockdown results in numerous cardiac defects. Images of whole mount (a-c) and haematoxylin- and eosin-stained frontal sections (d-f) of control and severe Tβ4shNk and Tβ4shMlc hearts at E14.5, to demonstrate epicardial nodules indicated by black arrowheads (b), pale, displaced ventricles (c) and failure in compaction of the ventricles (e, f). ra, right atrium; rv, right ventricle; la, left atrium; lv, left ventricle; oft, outflow tract; ivs, interventricular septum.

FIG. 3. Tβ4 promotes the differentiation of vascular smooth muscle and endothelial cells. Smooth muscle cell differentiation is delayed in Tβ4 knockdown hearts, indicated by the near absence of SMαA-positive cells in Tβ4shNk myocardium (my) and a considerable reduction in Tβ4shMlc myocardium, compared with control (a). Cultured EPDCs from E10.5 heart explants display maximum potential for differentiation into SMαA- and Tie2-positive cells (b); the potential for differentiation of these cell types diminishes by E12.5 (b). Tβ4 promotes differentiation of SMαA- and Tie2-positive cells, the latter additionally requiring VEGF and FGFs (b). VEGF may mediate aspects of Tβ4-induced coronary vessel formation. Expression of VEGF was reduced in the myocardium (my) of Tβ4shNk hearts, compared with the same region of control myocardium; white boxes indicate magnified region (c).

FIG. 4. Tβ4 is expressed in the myocardium and great vessels of the developing heart. RNA in situ hybridization (a-c) and immunohistochemical (d-f) detection of Tβ4 in wild-type E14.5 embryo. Tβ4 is expressed throughout the ventricular myocardium (a), robustly in the interventricular septum (ivs; b) and the myocardial compact layer (a). Further domains of Tβ4 expression in the vascular smooth muscle lining of the aorta (ao) and pulmonary artery (pa; c-f).

FIG. 5. Conditional knockdown of Tβ4 in ventricular cardiomyocytes. R26R crosses with Nkx2-5^{Cre}KI (a-c) and MLC2v^{Cre}KI (d-f) mice and X-gal staining to reveal Cre recombinase expression in cardiomyocytes of the ventricular myocardium (my), and exclusion from the epicardium (epi; c). Cre expression/X-gal staining is mosaic following crosses with either Cre knock-in strain 2 (a-f). Following a Nkx2-5CreKI cross expression is relatively widespread whereas expression is restricted to a proportion of ventricular cardiomyoctes following a MLC2v^{Cre}KI cross. The cohort of positive cells arising from a MLC2vCreKI cross express Cre at a higher level (d) relative to those from a Nkx2-5^{Cre}KI cross (a, b). Tβ4 is expressed throughout most cells of the ventricular myocardium (my; g) but is excluded from Flk1-positive endothelial cells (h, j).

FIG. 6. Tβ4 promotes migration of adult EPDCs and enables their differentiation into vasculogenic cells. Outgrowth of large colonies of cells from adult heart explants stimulated by Tβ4 (b, c), compared with a minimal degree of migration from untreated explants (a). Emerging cells (b, d, blue box) identified as epicardial cells by immunostaining for epicardin (e). Following migration, cells undergo differentiation into smooth muscle cells, identified by immunostaining for SMαA (b, d green box, g), fibroblasts identified by immunostaining for procollagen type I (b, d yellow box, f) and endothelial cells identified by immunostaining for Flk-1 (b, d red box, h).

FIG. 7. Disruption of the actin cytoskeleton, a direct consequence of Tβ4 knockdown, results in apoptosis of ventricular cardiomyocytes. Tβ4 knockdown led to severe disruption of the F-actin cytoskeleton, as determined by phalloidin staining (a-c). Fas activation (clustering) is greatly elevated in the myocardium (my) of Tβ4shMlc (e,f) and Tβ4shNk embryos; white arrowheads (h,i), compared with control (d,g). Initiator caspase-8 (j) and effector caspase-3 (k) are activated, resulting in increased apoptotic cell death, determined by TUNEL staining (l-n). TUNEL-positive cells are abundant in the epicardium (ep) and myocardium (my) and relatively scarce in the compact zone (cz).

FIG. 8. Tβ4 does not appear to directly regulate the survival kinase Akt. (a). Tβ4 activates Akt (P-Ser 473) within 10 min of treatment of C2C12 4 myoblasts (b). However, increased Akt activation was observed in Tβ4shMlc and Tβ4shNk hearts, arguing against a direct requirement for Akt activation in coronary vessel development.

FIG. 9. AcSDKP can be produced by peptidase cleavage of Tβ4 but not Tb10, the closely related b-thymosin expressed in the developing heart (a). AcSDKP levels are reduced in Tβ4shMlc (60%, P, 0.01, n55) and Tβ4shNk (62%, P, 0.01, n54) hearts as a result of Tβ4 knockdown (b). AcSDKP injection into pregnant females restored the peptide to control level in hearts of Tβ4shNk embryos (c). Restoration of AcSDKP in the developing heart is insufficient to rescue vascular defects associated with knockdown of Tβ4 (d); Tβ4shNk hearts with AcSDKP equivalent to control level display characteristic defects of severe Tβ4shNk knockdown hearts, such as the epicardial nodules highlighted by black arrowheads in (d). AcSDKP promotes the differentiation of adult EPDCs into Flk1-positive endothelial cells (e).

FIG. 10. Tβ4 and AcSDKP are up-regulated following myocardial infarction. Myocardial infarction induces an increase in endogenous Tβ4 (a) and AcSDKP (b) expression levels in the adult heart, determined by western blot and enzyme immunoassay, respectively.

FIG. 11. Tβ4 promotes vasculogenesis in the adult intact and injured heart. Western analyses (a, c) and immunohistochemistry (b, d) for vascular markers on adult intact (a, b) or infarcted (c, d) mouse hearts treated with either Tβ4 or vehicle (co). 28 day duration of treatment for intact hearts (time course of samples taken at 2, 4, 7, 14 and 28 days); 7 day duration of treatment post-MI (time course samples taken at 2 (d2), 4 (d4) and 7 (d7) days). Tie2, PECAM and VEGF are up-regulated in intact hearts following 2 days of Tβ4 treatment, accompanied by an increase in levels of SMαA and P-HH3 (a). After 28 days of Tβ4 treatment, PECAM and SMαA positive cells reveal new coronaries developing extensively throughout the sub-epicardial space and underlying myocardium compared to vehicle-treated controls (highlighted by black arrowheads; b). PECAM and SMαA are elevated post MI following 2 days of Tβ4 treatment compared to vehicle treated controls (c) and by day 7 post infarct PECAM and SMαA positive cells are observed surrounding the scarred myocardium (d). ep, epicardium; my, myocardium; sc, scar tissue; v, vehicle.

FIG. 12. Tβ4 treatment reduces myocardial scarring post-infarction. Equivalent plane of trichrome stained transverse sections from vehicle and Tβ4 treated hearts, 7 days post MI; red represents viable myocardium and blue represents collagen deposition indicative of scarring and fibrosis. Note the reduced scar volume, increased proportion of healthy cardiomyocytes and absence of pathological ventricular dilation in the Tβ4 treated heart compared to vehicle treated control.

FIG. 13. Immunofuorescence (a, b, d, f) and western analyses (c, e) for Isl-1 and Nkx2.5 and sarcomeric actin and cardiac myosin binding protein C (cMyBPC) as markers of cardiac progenitors and differentiated cardiomyocytes, respectively. Adult epicardial explants (a, b), intact adult hearts (c, d) and infarcted hearts (e, f) following Tβ4 treatment as compared to vehicle treated controls (co) for up to 28 days for the intact hearts and up to 7 days post MI for the injured hearts. After 24 hours in culture colonies of proliferative (Ki67 positive) cells emerge from epicardial explants which are double positive for Isl-1 and Nkx2.5 (a). Following removal of the explant and culture for a further 4 days, Isl1+/Nkx2.5+ cardiac progenitors differentiate into cardiomyocytes expressing sarcomeric actin and cMyBPC (b). Isl-1 and Nkx2.5 are elevated in adult hearts following 2 days of Tβ4 treatment (c) and Isl1+/Nkx2.5+ cells are observed emerging from the epicardium, migrating into the subepicardial space (d). Following MI Tβ4 treated hearts reveal an elevated expression of Isl1 and Nkx2.5 2 days post infarct which persists until day 4 (e). Tβ4 treatment results in significant numbers of Isl-1+/Nkx2.5+ cells (white arrowheads) in the subepicardial space 2 days post-infarct and after 7 days Isl-1+/Nkx2.5+ cells also localize to the border zone (indicated by the white dashed line) of the infarct (f). bz, border zone; co, control; ep, epicardium; my, myocardium; sc, scar tissue; v, vehicle.

FIG. 14. Immunohistochemistry on intact hearts for PECAM and SMαA following 7 days of Tβ4 or vehicle treatment. Whilst numbers of PECAM and SMαA positive cells (indicated by black arrowheads) are elevated in Tβ4 treated hearts compared to controls this is a relatively moderate response as compared to treatment following injury (see FIG. 11d).

FIG. 15. The upstream initiator of the acute inflammatory cascade TNF-α is down regulated at early stages post-MI (2 days) following Tβ4 treatment along with the downstream inflammatory cytokine IL-6; levels of both TNF-α and IL-6 subsequently become elevated after 7 days consistent with a reduction in injury early on and enhanced repair at later stages (a). Phospho-NFκB positive cells infiltrate infarcted myocardium after 2 days in control treated injured hearts (indicated by white arrowheads), as determined by immunofuorescence, whereas Tβ4 treatment significantly reduces the numbers of inflammatory cells located at the site of injury (b). The anti-inflammatory cytokine IL-10 is up-regulated after 2 and 4 days of treatment with Tβ4 along with the monocyte chemoattractant factor MCP-1, consistent with a reduction in acute inflammation (c). Following Tβ4 treatment the extracellular matrix is stabilized for appropriate repair as indicated by a down-regulation of MMP-9 and an upregulation of TIMP2 (d). ep, epicardium; my, myocardium; v, vehicle. Black arrowheads indicate alternate glycosylated isoforms (21 kDa and 24 kDa) of IL-10.

FIG. 16. Immunofluorescence (a, b) to demonstrate Tβ4-induced cardiac progenitors ex-vivo (a) which subsequently express markers of more differentiated cardiomyocytes (b). After 24 hours in culture, colonies of proliferating (Ki67+) cells emerge from adult epicardial explants (a, upper panels 1-4; white box in panel 1 is enhanced in panel 3; 10 μm scale bar in panel 4 applies to panels 2-4). Note the highlighted cells in panel 3 are all specifically Ki67+, consistent with the high level of proliferation as cells first emerge from the explant; cells cease to proliferate and begin to differentiate as they migrate away from the explant. Use of lineage-trace Gata5-EYFP adult hearts confirmed the epicardial origin of EYFP+ cardiac progenitors which express Isl-1 (panels 5-8), Nkx2.5 (panels 9-12) and Gata-4 (panels 13-16 with Tβ4 treatment (a; 5 μm scale bar in panel 16, applies to panels 5-16). Following removal of the explant and culture for a further 4 days, EYFP+ cardiac progenitors differentiate into cardiomyocytes expressing sarcomeric α-actinin (SαA) (b; panels 1-3), cardiac myosin binding protein C (MyBPC; panels 4-6) and cardiac troponin T (cTnT; panels 5-8) (b; 5 μm scale bar in panel 9 applies to panels 1-9).

FIG. 17. Western analyses (a, b) and immunofluorescence (c-j) to demonstrate Tβ4-induced myocardial regeneration using markers of cardiac progenitors and differentiated cardiomyocytes. Isl-1 and Nkx2.5 are elevated in intact adult hearts following Tβ4 treatment (a) and, following MI, Tβ4 treated hearts reveal an elevated expression of Isl1 and Nkx2.5, 2 days post-infarct which persists at day 4 (b). All western samples from control and Tβ4 treated hearts were run on the same gel/marker and separated for presentation (a). In vivo, lineage trace analysis revealed EYFP-positive epicardium-derived cardiomyocytes residing within the intact (not shown) and injured adult myocardium (c-j), EYFP+ cells at day 7 post MI, co-expressed cTnT (c) and were shown to be integrated with the resident myocardium as determined by the presence of connexin 43 (Cx43) positive gap junctions at low power (d-f). EYFP fluorescence and Cx43 staining followed by the merge for the same integrated cardiomyocytes are shown (d-f), note the comparable size of the EYFP+cardiomyoctes against resident (EYFP−) cardiomyocytes highlighted by the white scale lines in (f); arrowheads depict Cx43 staining of gap junctions (e, f). Higher power view of a cluster of EYFP+ cardiomyocytes, illustrating Cx43+ gap junctions, highlighted by white arrowheads (g). EPDC-derived cardiomyocytes were co-stained with two different polyclonal antibodies which recognise EYFP: α-YFP₁ (h, i) and α-YFP₂ (j) and SαA to confirm the EPDC origin of cardiomyocytes within resident myocardium (h-j). The white box in (h) is depicted at higher power in (i) to illustrate the striated appearance of the EYFP+/SαA+, integrated cardiomyocytes. Specificity of EYFP antibodies was ascertained by immunofluorescence on non-lineage trace hearts which detected neither EPDCs nor EPDC-derived cardiomyocytes (no signal; not shown). Cell quantification analysis revealed an endogenous myocardial regeneration response, indicated by the increased number of EYFP+ cardiomyocytes in the vehicle-treated injured heart (co) following MI (k, * p<0.1), a response which was significantly enhanced by Tβ4 (k, *** p<0.001). Rarely were EYFP+ cardiomyocytes detected in the border zone or scar tissue (<5% of total); the majority integrated within the myocardial wall proximal to the scar tissue (l). Tβ4 treatment resulted in a significantly increased number of EYFP+ cardiomyocytes in the proximal area compared with the remote myocardium (1, *** p<0.001 proximal, co v Tβ4; ** p=<0.01 Tβ4 proximal v remote). All p-values calculated by student's t-test. Schematic to illustrate myocardial regions in the left ventricle, proximal and remote to the site of injury (m). bz, border zone; co, control; ep, epicardium; LV, left ventricle; MI, myocardial infarction; my, myocardium; RV, right ventricle; sc, scar tissue; v, vehicle.

FIG. 18. Western analyses (a) and immunofluorescence (b-e) for vascular markers on adult intact mouse hearts treated with either Tβ4 or vehicle (co). Injection regimen: intraperitoneal injection of Tβ4 (150 μg in 0.1 ml PBS) or vehicle (0.1 ml PBS) was given every 2 days for up to 1 week or every 3 days for up to 4 weeks. Intact hearts were harvested after 2, 4, 7, 14 and 28 days and infarcted hearts after 2, 4 and 7 days post-MI. Tie2, PECAM and VEGF are up-regulated in intact hearts following 2 days of Tβ4 treatment, accompanied by an increase in levels of SMαA and P-HH3; western samples from control and Tβ4 treated hearts were run on the same gel/marker and separated for presentation (a). After 28 days of Tβ4 treatment, PECAM and SMαA positive cells reveal new capillaries developing extensively throughout the sub-epicardial space and underlying myocardium compared to vehicle-treated controls (highlighted by arrowheads; b-e); white asterisks in (b) and (c) denote background autofluorescence in the myocardium; inset of vessel in (c) illustrates specific PECAM staining of coronary endothelium.

FIG. 19. PECAM and SMαA protein levels are elevated post MI following 2 days of Tβ4 treatment compared to vehicle-treated controls and this increase in both PECAM and SMαA persists through to d7 post MI (a). After 7 days of Tβ4 treatment post infarct PECAM+ vessels (b) are observed surrounding the scarred myocardium; a comparatively reduced endogenous response to MI occurs even in control, as revealed by epicardial expansion and increase in PECAM+ vessels (c; white asterisk highlights autofluorescence background in the myocardium). A significant number of SMαA+ cells are observed delaminating from the epicardium and migrating into the underlying myocardium (highlighted by white arrow) following Tβ4 treatment (d) as compared with control-treated hearts where relatively few SMαA+ cells are observed highlighted by white arrowheads (e). Gata5-EYFP lineage trace analysis reveals EPDC association with and contribution to the coronary vasculature (f-h, grouped by black box; i). Clusters of small EYFP+ cells (green) were observed adjacent to, and in contact with, newly formed PECAM+ vessels (red) and merged panels show EYFP+ cells co-stained with PECAM (yellow; h; highlighted by white arrowheads). SMαA+ cells (green) contributed to EYFP+ vessels (α-EGFP to detect EYFP; staining in red), co-staining (yellow) reveals EPDC-origin of a proportion of the smooth muscle cells (i; co-stained cells highlighted by white arrowheads). Quantitative analysis of the number of rhodamine-conjugated dextran (Dextran-TRITC)-perfused coronary arteries in intact Cx40-EGFP adult hearts (j; lower two fluorescence panels) following 28 d treatment with Tβ4 or vehicle (co), illustrating a significant increase in stable vessels following Tβ4 treatment * p<0.01 (j), PECAM+ cell counts (k) on MI hearts following 4 d treatment with Tβ4 or vehicle (co) **p<0.001 and vessel area quantification (Image J; j) after 7 d treatment ***p<0.0001 (all p-values calculated by student's t-test). Following MI, an endogenous response induces new vessel formation which correlates with the extent of injury; the degree of new vessel formation is illustrated for a single example of MI at d7 which resulted in mild injury alongside a single example of a severe injury (I; error bars excluded since n=1 as representatives of each severity index). ca, coronary artery; co, control; cv, coronary vein; ep, epicardium; my, myocardium; sc, scar tissue; ses, subepicardial space; v, vehicle

FIG. 20. Proliferating (Ki67+) EPDCs emerging from explants treated with Tβ4 begin to re-express the fetal epicardial genes Tbx18, Raldh2 and epicardin (Epn) after 24 hours in culture, as detected by immunofluorescence (a, upper panels 1-12). Gata5-EYFP adult heart explant cultures confirm the epicardial origin of the cells and specificity of the epicardial markers (a, lower panels 13-24); 5 μm scale bar in panel 24 applies to all panels in (a). Tbx18 and Raldh2 protein levels are significantly up-regulated compared to controls in both the intact heart (b) and injured hearts (c) following 2 days of Tβ4 treatment. All western samples from control and Tβ4 treated hearts were run on the same gel/marker and separated for presentation (b). Post-MI, Tβ4 treatment for 2 days induces organ wide activation of the adult epicardium characterized by significantly elevated numbers of Tbx18+(d; highlighted by black arrowheads) compared to control vehicle-treated hearts (e). Raldh2+ (f) compared to controls (g); white asterisk highlights autofluorescence background in the myocardium (f, g) and WT-1+ cells (highlighted by white arrowheads; H, I) residing in the epicardium and subepicardial space. Accompanying DNA panels in (h) and (i) provide the absolute number of cells located in the epicardium and subepicardial space, within the fields of view, relative to those positive for WT-1. co, control; ep, epicardium; my, myocardium; v, vehicle.

FIG. 21. The upstream initiator of the acute inflammatory cascade, TNF-α, is down-regulated at early stages post-MI (2 days) following Tβ4 treatment along with the downstream inflammatory cytokine, IL-6; levels of both TNF-α and IL-6 subsequently become elevated after 7 days, consistent with a reduction in injury early on and enhanced repair at later stages (a). The anti-inflammatory cytokine IL-10 is up-regulated after 2 and 4 days of treatment with Tβ4 along with the monocyte chemoattractant factor, MCP-1; both IL-10 and MCP-1 are significantly reduced after 7 days of treatment consistent with the anti-inflammatory repair requirement at later stages (b); black arrowheads indicate alternate glycosylated isoforms (21 kDa and 24 kDa) of IL-10 (b). Consistent with MCP-1 up-regulation, Tβ4-treatment of MI hearts resulted in an enhanced infiltration of the injured myocardium with T helper (CD4+; c, d) or cytotoxic T (CD8+; e, f) lymphocytes (all of which were CD45+; g, h) after 4 days. This response was quantified by cell counts over a time course sampled at d2, d4 and d7 post-MI (i). Tβ4-induced leukocyte infiltration peaks at d4 but is rapidly cleared, returning to a level comparable with vehicle by d7 (i). Insets in (d), (f), (g) and (h) show high power views of the CD4+, CD8+ and CD45+ leukocytes, respectively, to illustrate specificity of the markers used for generating the cell counts data. 20 μm scale bar in (h) applies to panels (c-h). Macrophages (F4/80+ highlighted by white arrowheads; j-m) were elevated following Tβ4 treatment (j) at d2 post MI compared to vehicle treated control hearts (k) but were reduced at d7 post-MI (comparing l and m). Enhanced macrophage infiltration during the early acute phase (d2) of the inflammatory response followed by reduced macrophage presence during the more chronic phase (d7) was verified by cell counts illustrated in (n). Inset in (m) shows high power view of F4/80+ macrophage to confirm marker specificity for generating the cell counts data. 20 μm scale bar in (l) applies to panels (j-m). In keeping with the reduced expression of pro-inflammatory cytokines during the acute inflammatory phase (a), fewer cytotoxic myeloperoxidase (MPO)+ neutrophils are present in Tβ4-treated MI hearts, compared with controls (compare o with p; neutrophils in p are highlighted by white arrowheads), supported by cell counts (q) based on specific staining for MPO (inset in o). 10 μm scale bar in (o) applies to both panels (o) and (p). co, control; ep, epicardium; my, myocardium; v, vehicle; sc, scar.

FIG. 22. In Gata5-EYFP adult heart explants, emerging EPDCs are EYFP+ (a); differentiated EPDCs retain EYFP expression, as represented by a SMαA+ vascular smooth muscle cell (b-d); 2 μm scale bar in panel (d) applies to panels (b-d). Transverse sections of Gata5-EYFP adult mouse heart (e-j), EYFP fluorescence highlights cells residing in the outer epicardial layer and subepicardial space (e) and detects epicardial derivatives residing in the underlying myocardium (highlighted by white arrowheads; f) and coronary vessels (g); 40 μm scale bar in e applies to panel f. Lineage traced cells were stained with a mouse monoclonal α-EGFP antibody which recognizes EYFP (α-YFP) to exclude autofluorescence (h-j) and co-stained with either α-Tbx18 (i) or α-WT-1 (j) antibodies to reveal EYFP+ cells specifically in the outer epicardium and isolated cells co-expressing epicardial markers (highlighted by white arrowheads, i, j). 40 μm scale bar in h also applies to panels i and j. Following MI, an expansion of EYFP+EPDCs precedes their delamination from the epicardium (highlighted by white arrows) and migration towards the scar tissue (k); this response occurs even in the absence of Tβ4, as illustrated in a control heart (l-n; panels grouped by black box). 20 μm scale bar in panel (n) applies to panels (l-n). cv, coronary vessel; ep, epicardium; EPDCs, epicardium-derived cells; my, myocardium; sc, scar; ses, sub-epicardial space.

FIG. 23. Brightfield image of adult epicardial outgrowth containing immature progenitor-like cells⁵ after 48 hours of culture in the presence of Tβ4; presumptive progenitors are indicated by red arrowheads according to their primitive, rounded morphology.

FIG. 24. Immunofluorescence to identify Isl-1+ and Nkx2.5+ cardiac progenitors among the proliferating (Ki67+) cells that emerge from epicardial explants 24 hours after Tβ4 treatment; cardiac progenitors are double positive for Isl-1 and Nkx2.5 (low power views to illustrate relative incidence of cardiac progenitors; highlighted by white arrowheads; panels 1-8). Representative examples of individual cells under high power, which are either double positive for Isl-1 and Ki67 (panels 9-12), Nkx2.5 and Ki67 (panels 13-16) or Isl-1 and Nkx2.5 (panels 17-20), to highlight the fact that these myocardial progenitors exist in outgrowths following treatment with Tβ4. 20 μm scale bar in panel 8 applies to panels 1-8; 5 μm scale bar in panel 20 applies to panels 9-20.

FIG. 25. Western blots on heart extracts from 8 week old adult mice injected with vehicle (0.1 ml PBS) or Tβ4 (150 μg in 0.1 ml PBS) after 2, 4 and 7 days. Western blot analysis performed on heart extracts show that vascular (Tie2, PECAM, VEGF, SMαA), proliferation (P-HH3), early cardiac progenitor (Isl-1, Nkx2.5) and epicardial (Tbx18, Raldh2) markers were unchanged in vehicle-treated mice over the 7 day time course (a). Equivalent loading was ensured and normalized against GAPDH levels. Western blots showing the time course of vehicle treatment from 7-28 days and of Tβ4 treatment from 2-28 days are shown in FIGS. 2-5. Scanning densitometry, performed using Image J, as a quantitative representation of all western blots normalized against GAPDH levels. Histograms relate to western blots shown in FIGS. 2-5: FIG. 2a (b); FIG. 2b (c); FIG. 3a (d); FIG. 4a (e); FIG. 5b (f); FIG. 5c (g); FIG. 6a (h); FIG. 6b (i); grey and black bars indicate control and Tβ4 treatments, respectively. All scans are of single bands, except for the d2 MI westerns which include 3 bands per marker with S.E.M bars included accordingly (e, g).

FIG. 26. Equivalent plane of sections through the wall of the left ventricle stained with DAPI, 2 days post-MI (a, b) to illustrate the equivalent extent of scar size and loss of myocardium in vehicle- (a) versus Tβ4-(b) treated hearts prior to onset of myocardial regeneration; infarct is highlighted by white trace on duplicated lower panels in a and b; Image J analysis revealed respective infarct areas of 0.276 mm² (a) and 0.292 mm² (b); 100 μm scale bar in a also applies to b. Trichrome stained transverse sections (cut at the level of the ventricular papillary muscles) from vehicle- (c) and Tβ4-(d) treated hearts, 7 days post MI; red represents viable myocardium and blue represents collagen deposition indicative of scarring and fibrosis. Note the reduced scar volume, increased proportion of healthy cardiomyocytes and absence of pathological ventricular dilation in the Tβ4-treated heart (d) compared to vehicle-treated control (c). A degree of epicardial fibrosis is observed in both vehicle and Tβ4 treated hearts affecting up to 40% and 20% respectively of the epicardium. The remaining epicardium is intact and capable of responding to Tβ4 treatment, consistent with the pharmacological model and that previously reported⁸. ep, epicardium; lv, left ventricle; my, myocardium; pm, papillary muscle; sc, scar.

FIG. 27. Following MI, an increase in proliferation of cells (determined by Ki67 immunofluorescence) within the epicardium and subepicardial space was observed in Tβ4-treated hearts (a); highlighted by white arrowheads and shown at higher resolution in (b); very few Ki67+ nuclei were observed in the underlying myocardium (a) or the epicardium/sub-epicardial space of control hearts (c). Ki67+ nuclei were detected in EPDCs up to d7 post-MI, but were not detected at day 14 (not shown), indicating that proliferation occurs following MI as a component of the initial epicardial repair response. Co, control; ep, epicardium; my, myocardium; sc, scar tissue; ses, subepicardial space.

FIG. 28. Consistent with restoration of embryonic pluripotency to activated adult EPDCs, EYFP+ cells (a) co-stained with procollagen type I (b; highlighted by white arrowheads in c), indicative of fibroblasts, were detected in the expanded subepicardial space at d7 post-MI. 20 μm scale bar in panel (a) applies to panel (a-c). Fibroblast number did not vary significantly with Tβ4 treatment, compared with control: p=0.57; student's t-test (d).

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, particular methods and materials are now described. All publications mentioned herein are incorporated herein by reference to disclose and describe the methods and/or materials in connection with which the publications are cited.

### MODES FOR CARRYING OUT THE INVENTION

### Materials and Methods

### Western Blotting

Western blotting was performed using standard methods (Tris-Tricine 4-20% gradient SDS-PAGE for blotting of Tβ4 or Tβ10 peptides and Tris-glycine SDS-PAGE for all other proteins) using antibodies against Tβ4 (abcam), Tβ10 (Biodesign International), Tie-2 (Santa Cruz), SMαA (Sigma), GAPDH (Chemicon), Caspase-8 (Santa Cruz), Cleaved caspase-3, total Akt and Phospho-Akt (both Cell Signalling Technology).

HRP-conjugated secondary antibodies and ECL detection reagent were used to develop blots. Scanning densitometry was performed and quantified using Scion Image (Scion Corporation).

### Immunofluorescence

10 μm paraffin or cryostat sections were prepared for immunofluorescence using antibodies to SMαA (Sigma), Flk1 (BD Pharmingen), Fas, VEGF or Tie-2 (all Santa Cruz). Adult EPDCs were fixed in 4% PFA and incubated with antibodies against epicardin (TCF21, abcam), Flk1, SMαA or Procollagen type I (Santa Cruz). The following secondary antibodies were used: Cy3-conjugated anti-rabbit (Fas, Tie-2), TRITC-conjugated anti-mouse (VEGF, SMαA on embryo sections), FITC-conjugated anti-mouse (SMαA in EPDCs), Alexa 488-conjugated anti-goat (Procollagen type I) or Alexa 594-conjugated anti-rat (Flk1).

### Immunohistochemistry and Tunel Staining

E14.5 embryos were embedded in paraffin and sectioned at 10 μm for immunohistochemistry using a polyclonal anti-Tβ4 antibody (abcam) and developed using a standard streptavidin-HRP method. DNA fragmentation was detected by TUNEL assay according to the manufacturer's protocol (Promega).

### Immunodetection Methods

For in vivo studies, Western blotting, immunofluorescence and immunohistochemistry were performed using standard protocols with the following antibodies: Ki67 (Dako Cytomation), SMαA, sarcomeric α-actinin (Sigma), cardiac myosin binding protein C (E. Ehler), CD31/PECAM-1, CD4, CD8b (all BD Pharmingen), Nkx2.5, VEGF, Tie-2 (all Santa Cruz), GAPDH, Tbx18 (both Chemicon), Isl-1 (clone 39.4D5, Developmental Studies Hybridoma Bank), Tbx18, TNFα, IL-6, IL-10, GFP (full length—detects EYFP), MCP1, cTnT, F4/80, CD45, MPO, (all abcam), Living colours Av peptide, polyclonal and mouse monoclonal JL-8 antibodies (detect EYFP, Clontech) Cx43 (Zymed) and Raldh2 (gift of P. McCaffery). Images were acquired using either a Zeiss Axiolmager with ApoTome or a Zeiss LSM 510 confocal microscope equipped with argon and helium neon lasers using a 63×/1.4 lens.

### RNA In Situ Hybridization

RNA in situ hybridization was performed on paraffin-embedded sectioned embryos, as previously described [48] using a cDNA probe from the 3′UTR of Tβ4.

### X-Gal Staining of R26R x Cre Embryos

Embryos were equilibrated in 30% sucrose in PBS overnight at 4° C. and embedded in OCT medium. 15 μm cryostat sections were prepared, post-fixed in 4% PFA for 5 minutes and washed in PBS containing 2 mM MgCl2. Slides were incubated in X-gal tain solution (1 mg/ml 4-chloro-5-bromo-3-indolyl-β-galactosidase, 4 mM 4Fe(CN)6.3H2O, 4 mM K3Fe(CN)6, 2 mM MgCl2 in PBS) at 30° C. for 24 hours, insed in PBS and counterstained with 0.1% nuclear fast red (Sigma).

### Myocardial Infarction

Adult heart samples post myocardial infarction (MI) were kindly provided by James Clark, Cardiovascular Division, King's College London, St. Thomas' Hospital. Briefly, MI was induced in anaesthetised C57B1/6 male mice by ligation of the left anterior descending coronary artery for 30 minutes, followed by reperfusion. Animals were sacrificed one hour, one day or one week post MI and protein extracts prepared in Laemmli buffer for Western blotting and immunoassay to determine levels of Tβ4 and AcSDKP, respectively.

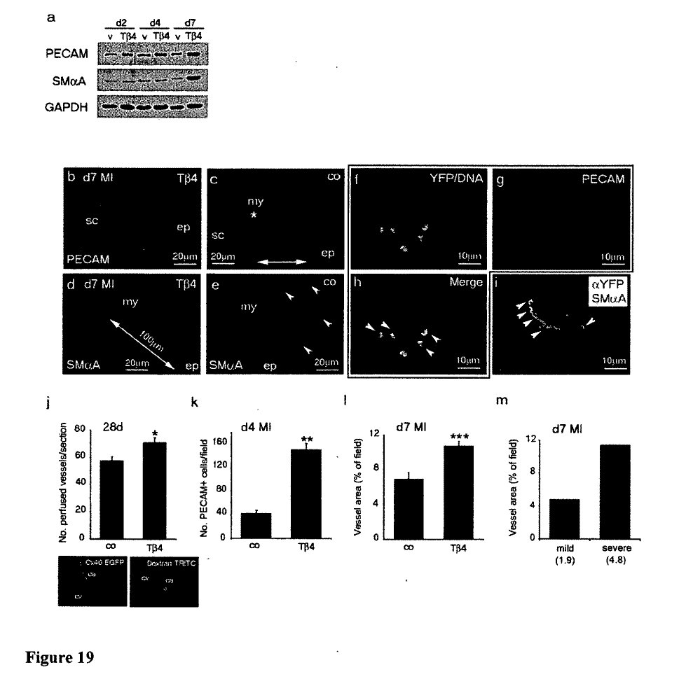

For in vivo studies, one hour after recovery, animals received intraperitoneal injection of Tβ4 (150 μg in 0.1 ml PBS) or vehicle (0.1 ml PBS) as previously reported [28]. Further injections were given after 2 and 4 days and hearts were harvested after 2, 4 and 7 days following ligation and prepared for western analysis and histological sectioning, as described above. Infarcts with equivalent extent of injury in the left ventricle were assessed for immunofluorescence and cell counts. Extreme examples of a mild and severe MI are included in FIG. 19m to demonstrate the correlation between vascular response and severity of injury.

### Methods of Obtaining EPDCs

A simple protocol for the outgrowth and differentiation of vasculogenic precursor cells (endothelial and vascular smooth muscle cells) from adult heart using Thymosin β4 to stimulate epicardial cell migration is described below.

### Reagents

Animals: 8-12 week old adult mice (C57B1/6 strain used; other strains not tested).
0.1% gelatin solution: prepare 0.1% (w/v) gelatin (Sigma Cell Culture G-1890) in distilled water; sterilize by autoclaving
1×DPBS (Invitrogen 14190-094)
DMEM+GlutaMAX™-1 (+4.5 g/L glucose, -pyruvate; Invitrogen 61965-026)
Foetal Bovine Serum (EU approved origin, Invitrogen 10106-169)
Penicillin-streptomycin solution, 10,000 units/ml penicillin G sodium, 10,000 ug/ml streptomycin sulphate; Invitrogen 15140-122)
Thymosin β4 (Immundiagnostik, Germany) 1 mg
4% paraformaldehyde in PBS (diluted from 37% solution, Sigma)
BLOCK: 10% sheep serum (Sigma) and 1% BSA (Sigma) in PBS for blocking non-specific binding of antibodies
Primary antibodies of choice, such as epicardin (TCF21, abcam), SMalphaA (Sigma), Flk1 (BD Pharmingen), or Procollagen type I (Santa Cruz)
Appropriate secondary antibodies: Cy3-conjugated anti-rabbit (epicardin; abcam) FITC-conjugated anti-mouse (SMαA; DAKO), Alexa 488-conjugated anti-goat (Procollagen type I; Invitrogen Molecular Probes) or Alexa 594-conjugated anti-rat (Flk1; Invitrogen Molecular Probes)
Hoechst 33342 (5 ug/ml in PBS)
50% glycerol in PBS

### EPDC Culture Medium

Supplement DMEM (containing GlutaMaxI and 4.5 g/L glucose) with the following: 15% FBS; 100 units/ml penicillin; 100 μg/ml streptomycin. CRITICAL: Prepare fresh medium, store at 4° C. and replace at least every month. Do not supplement with Tβ4 until ready to use.

### Thymosin β4

To prepare 1000× stock (10 ug/ml): dilute 1 mg stock into 10 ml sterile DPBS. Aliquot and store at −80° C. until required. Avoid repeated freezing and thawing. When required, dilute 1 μl to each ml of EPDC culture medium (final concentration 100 ng/ml), immediately prior to use.

### Equipment

Scalpel blade, forceps and dissection scissors, sterilized in 70% ethanol for 5 minutes. Blot excess ethanol and allow to air dry inside the sterile culture hood before use.
Tissue culture plates: 35 mm diameter, 6 well (recommended for culture; other sizes may be used but require optimisation of amount of heart tissue and medium to be used. Nunc or Becton Dickinson).
Optional: glass coverslips (18×18 mm or 18 mm diameter). Recommended if cells are to be analysed by immunofluorescence.
Fluorescence microscope (such as Axio Imager, Zeiss).

### Preparation of EPDCs.

1. Coat 6-well plates with gelatin: pipette 2 ml 0.1% gelatin solution, allow to stand for 15 minutes and aspirate. Optionally, place coverslips into wells, prior to gelatin coating.
2. Cull adult mouse by cervical dislocation.
3. Using sterile forceps and scissors make a lateral incision in the centre of the abdomen and tear back the fur to expose the rib cage.
4. Carefully cut upwards through the sternum and along the diaphragm, taking care not to cut into the heart. Pull back the ribs to reveal the heart.
5. Remove the heart using forceps and dissect away the atria and major vessels, to leave right and left ventricles.
6. Place tissue in a 60 mm tissue culture dish (non gelatin coated) containing 2 ml DPBS. Cut into quarters and allow blood to rinse from the tissue. Carefully aspirate away DPBS. Using a sterile scalpel, cut the heart into pieces of approximately 1 mm³. Reproducible EPDC outgrowth strongly depends upon the size of the heart pieces (optimally 1 mm³). Larger pieces will not adhere to permit sufficient migration while smaller pieces tend to dissociate completely and cardiomyocyte death precedes adherence and EPDC outgrowth.
7. Divide heart pieces into 4 equal portions (1 adult heart is typically divided between 4 wells for optimal EPDC outgrowth).
8. Pipette 2 ml of EPDC culture medium, supplemented with 100 ng/ml Tβ4, into each well to be used.
9. Place 1 portion of heart tissue into the centre of each well and ensure that all pieces are submerged.
10. Gently transfer the plate to a humidified 5% CO2 37° C. incubator. Maintain cultures with minimum disturbance to allow explants to adhere. No feeding is required for the first 72 hours. Minimal disturbance is absolutely essential for EPDC outgrowth. Explants adhere only tenuously in the first instance and disturbance in the first few days of culture will prevent adhesion or lead to detachment. Plates should be transferred extremely cautiously between incubator and microscope or culture hood. After sufficient EPDCs have emerged, explants attach more firmly but care is still required as detachment may easily occur.
11. After 72 hours in culture, carefully transfer plate to culture hood, wash explants gently with DPBS and add 2 ml fresh EPDC medium containing 100 ng/ml Tβ4. Leave for a further 24 hours before assessment of cellular phenotype.

### Characterisation of EPDC Phenotypes by Immunofluorescence.

1. Culture adult heart explants as described above.
2. After 72 hours of culture, fix cells with 4% PFA for 10 minutes at room temperature.
3. Wash cells twice with PBS.
4. Permeabilise cells with 0.5% Triton X-100 in PBS for 5 minutes at room temperature.
5. Wash cells twice with PBS.
6. Block non-specific binding by incubating cells in BLOCK (1% BSA/10% sheep serum in PBS) for 1 hour at room temperature.
7. Incubate cells with an appropriate dilution of primary antibody (epicardin, 1:100; SMalphaA, 1:700; Flk1, 1:100), or Procollagen type I, 1:100), in BLOCK.
8. Wash cells 3 times using BLOCK.
9. Incubate cells with the appropriate secondary antibody (epicardin: Cy3-conjugated anti-rabbit; SMalphaA: FITC-conjugated anti-mouse, 1:30; Procollagen type I: Alexa 488-conjugated anti-goat, 1:200; Flk1: Alexa 594-conjugated anti-rat, 1:200) diluted in BLOCK.
10. Wash cells twice in PBS.
11. Optionally, to stain nuclei, incubate with 5 ug/ml Hoechst in PBS for 5 minutes at room temperature.
12. Wash cells twice in PBS.
13. Mount coverslips onto microscope slides using 50% glycerol in PBS as mountant and visualize using a fluorescence microscope.

### Adult Epicardial Explant Cultures

Adult EPDCs were prepared from 10 week old C57B1/6 or Gata5-EYFP mice in the presence or absence of Tβ4 (100 ng/ml), as described above and at (http://www.natureprotocols.com/2006/11/17/thymosin_{—}4_t4induced_outgrowth.php). After 24 hours cells were either fixed in 4% paraformaldehyde (PFA) or explants were removed and cells that had migrated from the explant were allowed to differentiate for 4 days in DMEM containing 15% FBS, prior to fixing in 4% PFA.

### Culture and Tβ4 Treatment of C2C12 Myoblast Cells

C2C12 cells were cultured in DMEM containing 10% FBS. Tβ4 (10 ng/ml, mmundiagnostik AG) was added and cells harvested over a time course to assess the degree of phosphorylation (activation) of Akt.

### Transgenic Animals

### Conditional Knockdown of Tβ4 in the Developing Heart

The conditional RNAi approach was adopted following in vitro studies which demonstrated a putative role for Tβ4 in regulating cytokinesis and consequently cell survival (data not shown). Since Tβ4 maps to the X chromosome in the mouse, targeting of Tβ4 using either conventional or conditional approaches in ES cells could result in either a complete or partial loss of Tβ4 function respectively, and ultimately a failure in ES cell survival. Moreover, the use of conditional RNAi provided the possibility of generating a phenotypic range (dependent upon transgene copy number and insertion site), equivalent of a hypomorphic allelic series, for dissecting out Tβ4 function in the heart.

Construction of Tβ4 shRNA Transgene

The Tβ4 shRNA construct was prepared by modifying a RasGAP shRNA transgene, kindly provided by G. Gish (S.L.R.I., Toronto). The RasGAP shRNA sequence was removed and replaced with sense and antisense Tβ4 Sequences of 21 base pairs in length, separated by a nine bp spacer, downstream of the H1 RNA pol III promoter, followed by a stretch of five thymidines which act to terminate transcription. A 5-thymidine stop termination sequence, flanked by 2 loxP recombination sequences, was inserted after the H1 RNA pol III promoter, upstream of the 21-mer Tβ4 hairpin sequences. Thus, in the absence of Cre recombinase, transcription will ordinarily be terminated prior to synthesis of the Tβ4 shRNA and Tβ4 expression unaffected. Transgenic mice were derived by genoway (France) using standard procedures.

Cardiac-Specific Knockdown of Tβ4

To investigate a role for Tβ4 during heart development and to provide insight into the mechanism by which the peptide mediates adult cardiac repair, we generated mouse embryos with heart-specific Tβ4 deficiency using a novel strategy of transgenic conditional RNA interference (RNAi; as described above). Floxed Tβ4 short hairpin RNA (Tβ4shRNAflox) mice were crossed with two strains of Cre-expressing mice: Nkx2-5^{Cre}KI (designated Tβ4shNk), which directs Cre expression throughout the majority of cardiomyocytes [49, 50], and MLC2vCreKI (designated Tβ4shMlc), which directs Cre expression specifically to ventricular cardiomyocytes [51]. Tβ4shNk embryos were also observed to have thymic defects consistent not only with Cre expression driven by Nkx2-5 in the developing thymus [49], but also with the thymus representing an obvious source of Tβ4.

### Generation of Epicardium- and EPDC-Restricted Gata5-EYFP Lineage Trace Mice

Gata5-EYFP mice were generated by crossing the Gata5-Cre transgenic strain [19] with homozygous R262R EYFP reporter mice [25] and genotyped as previously described [19, 25].

### Gain of Function: Tβ4 Administration

Wild type (WT) C57B1/6 or Cx40-EGFP [46] male mice (25-30 g) received intraperitoneal injection of Tβ4 (150 μg in 0.1 ml PBS) or vehicle (0.1 ml PBS) every 2 days for up to 1 week or every 3 days for up to 4 weeks. Doses were based on previous studies [28, 46]. WT hearts were harvested after 2, 4, 7, 14 and 28 days and bisected transversely; the apex was snap frozen for protein preparation and the remaining tissue was fixed in 4% PFA for 2 hours for cryosectioning. Cx40-EGFP hearts were harvested after 7, 14 and 28 days and fixed, as above, for cryosectioning. Prior to harvest at the 28 d time point, Cx40-EGFP mice were intravenously injected with rhodamine-conjugated dextran (70 kDa, Invitrogen) at 2 mg per 20 g body weight, to assess coronary vessel perfusion.

### Results

### Thymosin β4 is Required for Coronary Vessel Development

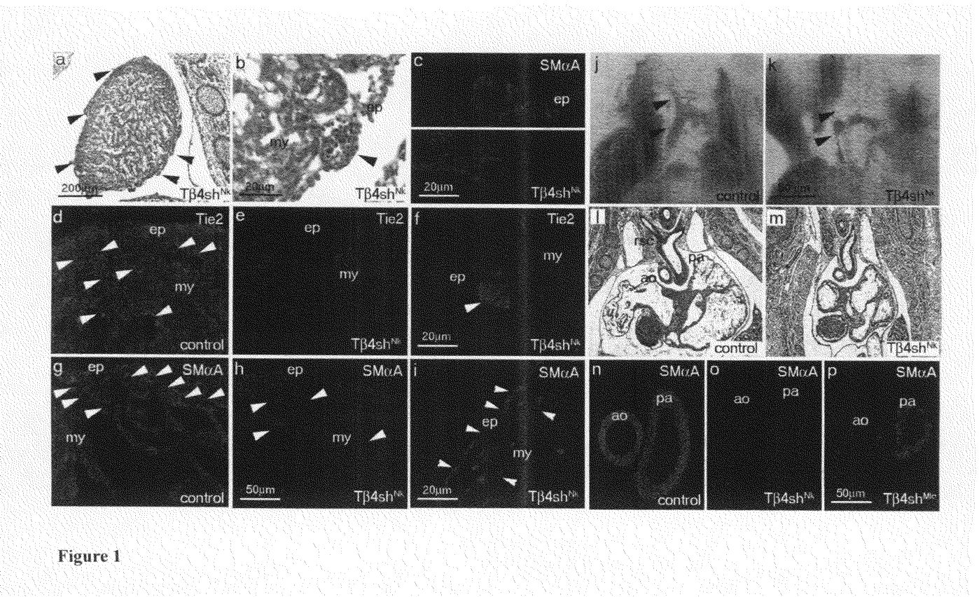

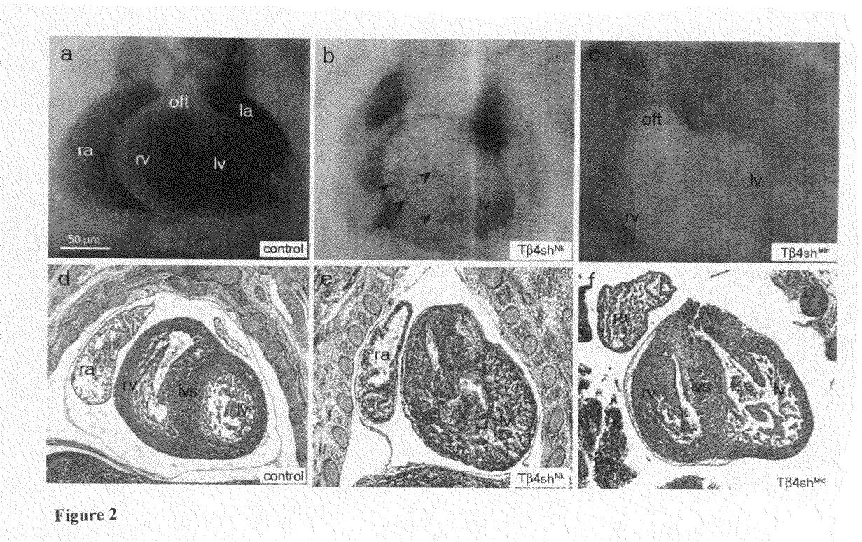

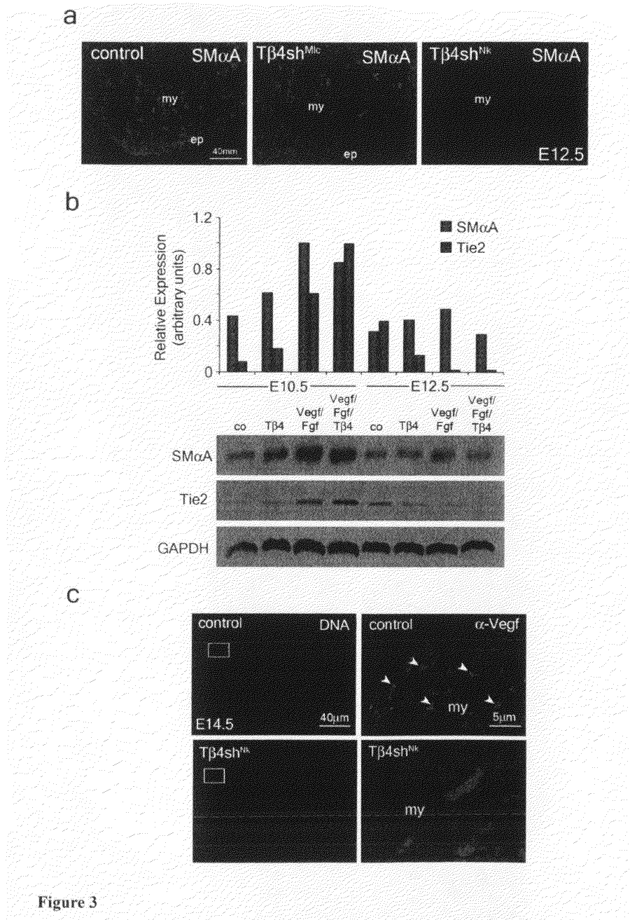

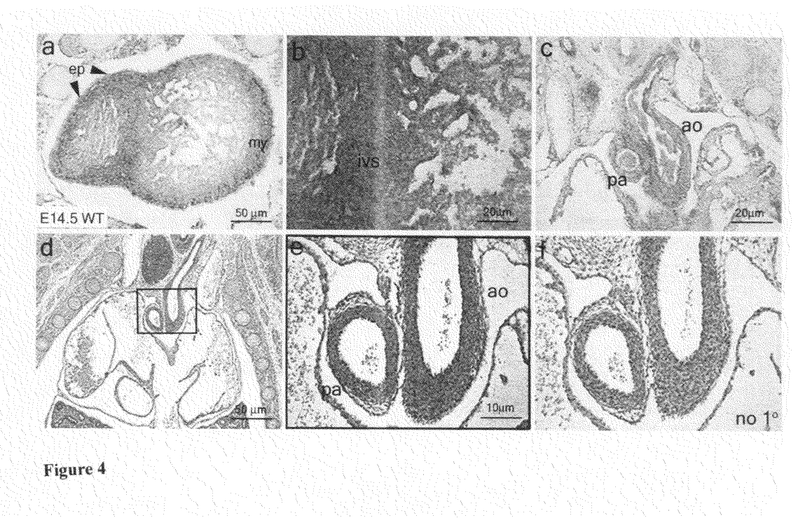

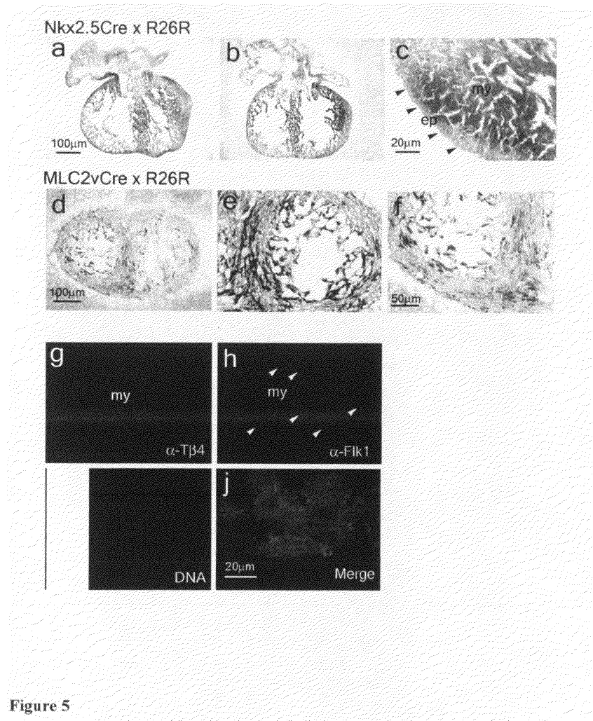

The epicardial nodules in Tβ4shNk embryos at E14.5 were cannular, composed of a thin endothelial layer containing a few pericytes, and blood-filled (FIG. 1 a to c). Micro-vessels lined with cells positive for the endothelial specific receptor, Tie2 [52], could be seen invading the dense myocardium of control hearts (FIG. 1 d); in contrast, the disrupted myocardium of Tβ4shNk embryos was almost entirely negative for Tie2, with only a few malformed vessel-like structures apparent (FIG. 1 e). Significantly, the epicardial nodules of Tβ4shNk embryos were intensely stained with Tie2, in clear contrast to the weak expression in themyocardium (FIG. 1 f). Coronary vasculogenesis requires that cells delaminate from the epicardium, undergo epithelial-mesenchymal transformation, migrate towards the capillary plexus within the myocardium and differentiate into endothelial cells [53]. The aberrant nodules in the Tβ4shNk mutants represent a population of Tie2-positive epicardium-derived cells (EPDCs) that have attempted, but failed, to migrate through the myocardium to form coronary vessels. Consistent with impaired vessel development were the thin myocardium and failed ventricular compaction (FIG. 2e, FIGS. 1 a and e), a process known to be dependent on epicardially-derived vasculogenesis [54]. Vascular progenitors in the developing epicardium display bipotency. EPDCs can either form endothelial cells, in response to a combination of myocardial vascular endothelial growth factor (VEGF) and basic fibroblast growth factor (βFGF) signalling, or differentiate into smooth muscle cells, upon exposure to platelet-derived growth factor (PDGF) and transforming growth factor β (TGFβ) [55, 56]. Therefore, we next investigated whether there was a reduced incidence of smooth muscle cell recruitment to nascent vessels in Tβ4 knockdown hearts. In controls, smooth muscle cells, detected by immunostaining for smooth muscle α-actin (SMαA), were evident throughout the myocardium specifically surrounding the lumen of micro-vessels (FIG. 1 g), whereas in Tβ4shNk hearts the majority of SMαA-positive cells persisted in the epicardium and in the reduced compact layer, rarely localized to the inner trabeculae (FIGS. 1 c, h, and i). At earlier stages (E12.5), SMαA-positive cells were barely detectable in the myocardium of Tβ4shNk mutants and only sparsely distributed in Tβ4shMlc mutants compared to controls (FIG. 3a). This suggests that, as a consequence of reduced Tβ4 signalling from the myocardium, there is a significant delay in smooth muscle cell differentiation. Moreover, EPDCs, fated to form smooth muscle cells, fail to migrate extensively into the myocardium to provide support to the coronary vessels and instead activate the smooth muscle cell differentiation program in situ. Defective recruitment of smooth muscle cells, so-called collateral growth [56], was also observed in the apical media of the large thoracic vessels of Tβ4shNk (and to a lesser extent, Tβ4shMlc) embryos. Both aorta and pulmonary artery were morphologically abnormal and visibly defective in terms of both angiogeniesis and arteriogenesis, with decreased vessel wall stability and hemorrhaging (FIG. 1 j to p). Failed contribution of EPDC-derived smooth muscle cells may explain in part the loss of SMαA-positive cells from the mutant thoracic vessels (FIG. 1 o and p); further loss probably arises as a consequence of failure of the coronary vasculature to establish appropriate collateral connections to the aortic root [56]. The defects associated with Tβ4 knockdown in the myocardium are clearly non cell-autonomous. Myocardial compaction, coronary vasculogenesis and smooth muscle cell recruitment all relate to the epicardial lineage, and specifically to EPDC differentiation and appropriate migration [53. 52]. Tβ4 is expressed throughout the developing myocardium, but is expressed neither in the epicardium (FIG. 4) nor in endothelial cells residing specifically within the wall of the heart at equivalent stages (FIGS. 5 g to j). Therefore, knockdown of intracellular Tβ4 in the myocardium results in loss of functional secreted Tβ4 and impaired paracrine signalling to the epicardium. Myocardial signalling associated with coronary vessel development from epicardium has also been attributed to the GATA co-factor friend of GATA 2 (FOG-2) [57] and to VEGF [58]. However, FOG-2 and VEGF are implicated exclusively in the induction of coronary vasculogenesis, whereas Tβ4 uniquely affects all aspects of coronary vessel development.

Tβ4 Promotes Neovascularisation from Embryonic Epicardium

In order to support our in vivo phenotype analyses and assess a direct effect of Tβ4 on developing epicardium, we established epicardial explant cultures from wild-type hearts13. We initially derived epicardial explants from stages E10.5 to E16.5 and postnatal day 1 (P1) neonates, treated with either, Tβ4, with VEGF and FGF7 [59], or a combination of Tβ4 and growth factors. Explants from E10.5 hearts, coincident with the formation of the epicardium, produced extensive outgrowths that differentiated into SMαA- and Tie2-positive cells (FIG. 3 b). Addition of Tβ4 significantly increased the numbers of SMαA- and Tie2-positive cells, and these cell populations were enhanced further still with the addition of VEGF and FGF7 (FIG. 3 b). The most potent effect was observed following combined treatment of Tβ4 and VEGF/FGF7, which resulted in a highly significant increase in Tie2-positive endothelial cells. Synergistic function of Tβ4 with VEGF in this assay is consistent with our observed downregulation of VEGF in Tβ4 knockdown hearts (FIG. 3 c), suggesting appropriate VEGF expression may require Tβ4. The ability of the epicardial explants to produce outgrowths of cells competent to differentiate in response to treatment significantly diminished over the course of development as observed at E12.5 (FIG. 3 b), and continued to decrease such that by E16.5 and P1 neonate stages there was no detectable expression of either SMαA or Tie2 (data not shown). This suggests that isolated epicardial derived cells after emerging from the epicardium have an optimal window of time in development (E10.5-12.5) during which they can respond to myocardial signals and differentiate into vascular progenitors.

### Tβ4 Induces Adult Epicardial Cell Migration

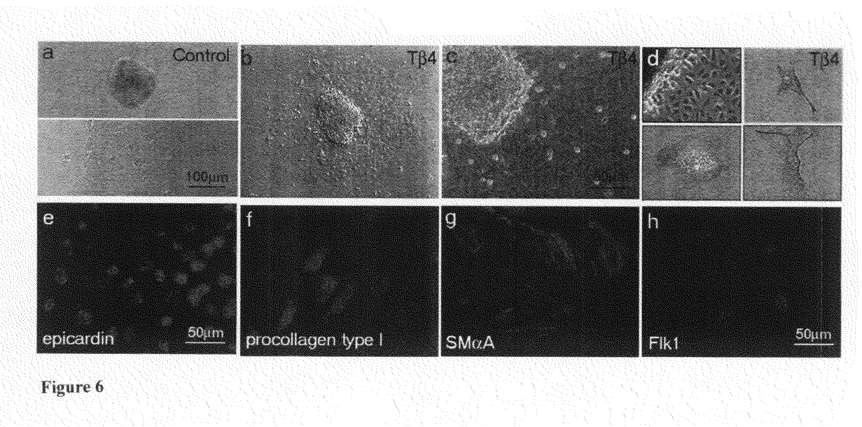

As coronary vasculogenesis is required to maintain cardiomyocyte survival and consequently appropriate myocardial architecture and cardiac function, the role of Tβ4 in coronary vessel development may underlie its reported ability to act therapeutically in terms of cardioprotection and repair [28]. Translation of a vascular development role for Tβ4 to that of angiogenic therapy for coronary artery disease in the adult heart requires releasing the adult epicardium from a quiescent state and restoring its pluripotency. To investigate the potential for Tβ4 in this context, we isolated epicardial explants from wildtype adult mouse hearts at 8-12 weeks of age (FIG. 6), and assessed the ability of Tβ4 to induce outgrowth in addition to any differentiation phenotype. Untreated adult explants demonstrated virtually no detectable outgrowth (FIG. 6 a), with only a few isolated cells observed in the culture dish (FIG. 6 a). This is consistent with the adult epicardium having lost migration, differentiation and signalling capacities during the latter half of gestation [60]. In contrast, treatment with Tβ4 stimulated extensive outgrowth of cells that differentiated into a variety of discernable phenotypes (FIG. 6 b-d). The emerging epithelial cells were positive for the epicardial-specific transcription factor epicardin (FIG. 6 e), and these cells differentiated with migration into procollagen type I—, SMαA- and Flk1 (VEGF receptor)-positive cells indicative of fibroblasts, smooth muscle and endothelial cells (FIG. 6 f-h). EPDCs account for a significant proportion of the 50% of cells in the murine cardiac wall that are not cardiomyocytes [61]; their contribution is assumed to be confined entirely to embryonic epicardium. Remarkably, the explant studies reveal for the first time an equivalent potential within the adult lineage. Vascular regeneration includes adaptive vasculogenesis and arteriogenesis [62], and the supply of endothelial and smooth muscle vascular precursors required for this process has been attributed, in part, to the peripheral circulation and bone marrow [63, 64]. Here we demonstrate the enormous potential of the adult epicardium under the control of Tβ4. Release of quiescent EPDCs represents a viable source of vascular progenitors for continued renewal of regressed vessels at low basal level or sustained neovascularization following cardiac injury. In ischaemic and failing hearts, Tβ4-treated adult EPDCs have the potential to deliver endothelial, smooth muscle and fibroblastic cells to sites of injury that need additional sustenance, thus ensuring adequate perfusion of damaged heart muscle and structural integrity of the myocardium. Tβ4 dependent vascularization underlies cardiomyocyte survival.

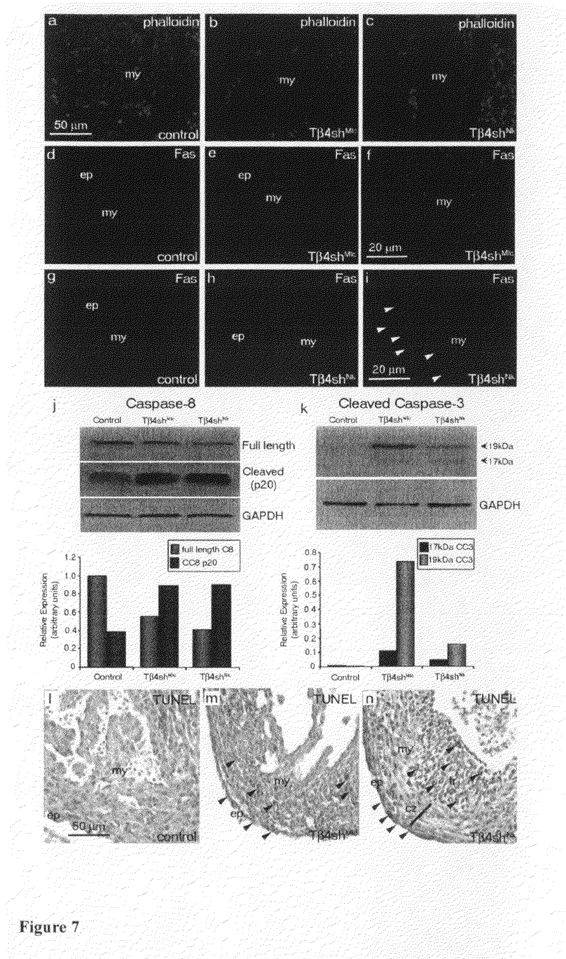

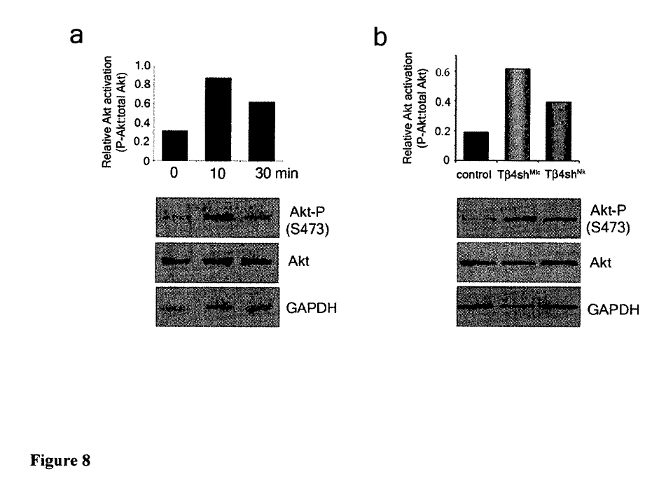

A major consequence of Tβ4 knockdown in the mutant hearts was a failure to maintain an integrated actin cytoskeleton, as revealed by global disruption of F-actin (FIG. 7 a-c). A high proportion of cells in the myocardium of both Tβ4shNk and Tβ4shMlc mutant hearts showed Fas clustering at the cell membrane, indicative of Fas activation (FIG. 7 d-i), increased production of the cleaved (active) forms of initiator caspase-8 and effector caspase-3 consistent with induction of an apoptotic cascade (FIG. 7 b), and elevated TUNEL (TdT-mediated dUTP nick end labelling) staining as evidence of increased DNA fragmentation (FIG. 7 l-n). This supports a role for Tβ4 in maintenance of cardiomyocyte survival, previously thought to be due to Tβ4-induced activation of the survival kinase and anti-apoptotic factor, Akt (also known as protein kinase B (PKB) [28]. We confirmed early activation of Akt in C2C12 (mouse myoblast) cells treated with Tβ4 as previously described2 (FIG. 8 a), but contrary to expectation active Akt-P (Ser 473) was not downregulated and was significantly higher in Tβ4shNk and Tβ4shMlc hearts than in control hearts (FIG. 8 b). From a cell survival perspective this finding suggests that Tβ4 is unlikely to directly regulate Akt and that Akt activation may be a secondary response to Tβ4 treatment, or that the increased activation of Akt may be a compensatory mechanism in the absence of Tβ4.

AcSDKP Stimulates Endothelial Cell Differentiation from Adult Epicardium

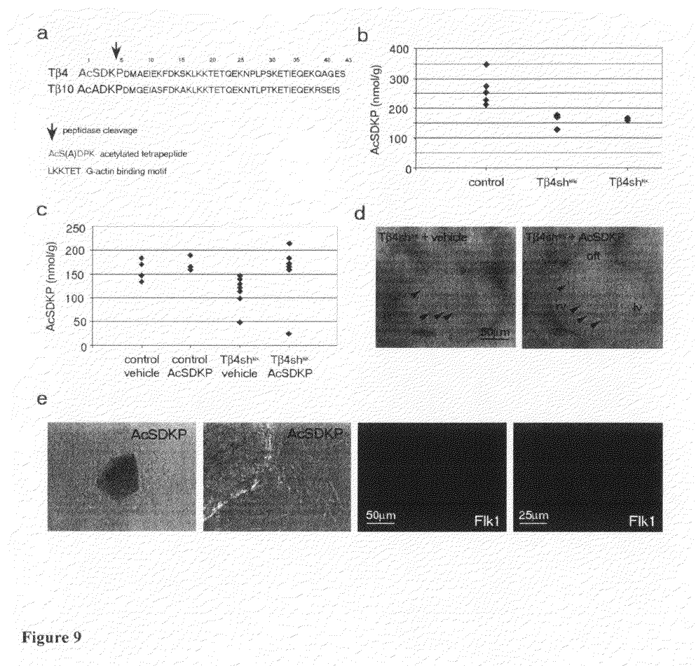

In our model, preservation of myocardium is secondary to Tβ4-induced coronary vasculogenesis, angiogenesis and collateral growth. The mechanism by which Tβ4 stimulates coronary vessel development in this regard involves Tβ4 directly promoting EPDC migration from the epicardium via its previously known function of actin binding, filament assembly and lamellipodia formation. However, scope exists for a non actin-mediated vasculo-, angio- and arteriogenic function for Tβ4 by virtue of its endoproteinase activity to produce the pro-angiogenic tetrapeptide N-acetyl-seryl-aspartyllysyl-proline (AcSDKP; FIG. 9a) [65 to 68]. We therefore quantified AcSDKP levels in our mutant knockdown hearts by competitive enzyme immunoassay on extracted myocardium, and found they were decreased to 62% and 60%, respectively, of that of controls (FIG. 9b, P, 0.01); this is robust evidence for a peptide and precursor peptide relationship between Tβ4 and AcSDKP in a physiological setting.

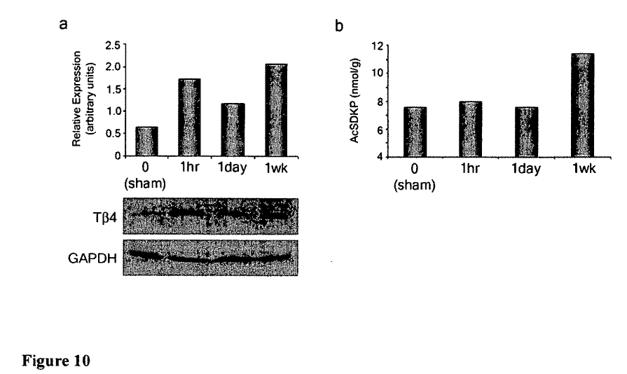

We next investigated whether AcSDKP could rescue any of the vasculogenesis defects observed in the Tβ4 mutant hearts. Intraperitoneal injection of AcSDKP into pregnant females successfully restored the peptide to control levels in mutant embryo hearts at E14.5 (FIG. 9c), but failed to rescue any of the phenotype attributed to Tβ4 knockdown (FIG. 9d). This supports our interpretation of the primary phenotype in Tβ4 mutants. AcSDKP lacks actin binding function, rendering it unable to stimulate filamentous actin assembly and lamellipodia-based cell migration and consequently unable to rescue the EPDC defect. However, in the adult, consistent with reported cardioprotective effects of AcSDKP [69, 70], we observed a significant upregulation in levels of both endogenous Tβ4 and AcSDKP in response to ischaemia after 1 day and 1 week, respectively (FIG. 10). Moreover, addition of AcSDKP to adult epicardial explants resulted in a striking increase in differentiated (Flk1-positive) endothelial cells (FIG. 9 e). Although unable to promote epicardial outgrowth beyond control levels, AcSDKP brought about rapid differentiation of any emerging EPDCs, such that Flk1-positive cells were observed in close proximity to the explant tissue (FIG. 8e). The differentiated cells were almost exclusively endothelial, with only very few smooth muscle cells observed in AcSDKP-treated cultures (data not shown). This suggests that cleavage of AcSDKP from Tβ4 exclusively promotes EPDC endothelial cell differentiation, and may underlie a compound vasculogenic effect of Tβ4 aside from simply promoting EPDC migration into overlying myocardium as an instructive cue for differentiation. Crucial to the further understanding of this two-step function will be the identification of the respective receptors for Tβ4 and AcSDKP.

### Neovascularisation

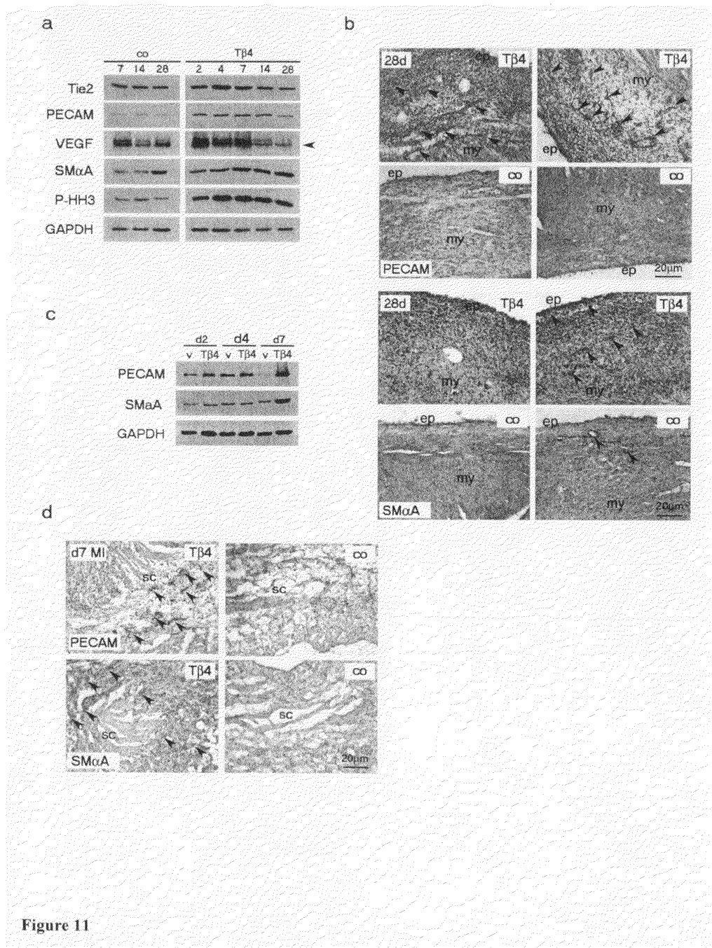

The minimum requirement for Tβ4 to promote EPDC-derived vascular endothelial and smooth muscle cells, was reported previously [26]. To investigate whether Tβ4 could stimulate new vessel growth in vivo, we established a gain of function mouse model by intraperitoneal injection of Tβ4 or vehicle (injections of 150 μg in 0.1 ml PBS every 2 days for first 4 days and every 3 days thereafter) into wild type 8 week old adult mice. Hearts were examined by western analysis and immunohistochemistry (IHC) for vascular markers and evidence of new coronaries at 2, 4, 7, 14 and 28 days post-treatment. Endothelial markers Tie2, PECAM and VEGF and the smooth muscle marker SMαA were significantly increased following 2 days of Tβ4 treatment compared to controls (FIG. 1a). This increase persisted throughout the duration of the experiment and was accompanied by a significant increase in proliferation as determined by elevated levels of phosphor-histone H3 (P-HH3; FIG. 1a). Immunostaining of treated hearts showed strong regionalised staining for PECAM and SMαA in the subepicardial space and immediate underlying myocardium at 7 days post-treatment and subsequently an increase in the number of coronary vessels after 28 days as indicated by endothelial cell lined arterioles surrounded by smooth muscle (FIG. 11b). In addition nuclear counter staining revealed a highly significant increase in cell proliferation coincident with sites of neovascularisation (FIG. 1b). This suggests that not only can Tβ4 stimulated EPDCs promote neovascularisation in vivo but that this response can occur in the absence of injury.

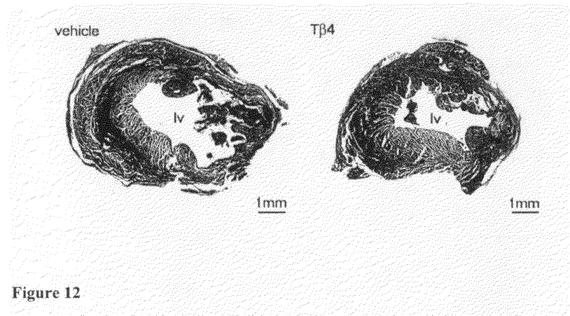

We next investigated whether Tβ4 could promote neovascularisation in vivo after myocardial damage and whether this may be enhanced as compared to the intact gain of function model. We established myocardial infarctions in adult mice, by coronary artery ligation, treated with either Tβ4 or vehicle (injections regimen as for gain of function; FIG. 12) and sampled the hearts for western and IHC analyses after 2, 4 and 7 days. Tβ4-treated infarcted hearts had significantly increased vascular endothelial and smooth muscle protein expression (FIG. 11 c) [which exceeded that observed in a non-injury setting (compare FIGS. 11 a and c)], equally up-regulation in vascular markers associated with the site of injury was significant (FIG. 11 d) and elevated beyond that observed in the subepicardial space of the intact gain of function hearts (compare FIGS. 11 b and d), consistent with optimal Tβ4-induced neovascularisation in the injury setting.

### Myocardial Regeneration

To-date a bona fide source of resident progenitor cells in the adult mammalian heart which may give rise to de novo cardiomyocytes with the potential to replenish injured muscle post-infarction has yet to be identified.

In the avian embryo EPDCs have been shown to differentiate into cardiomyocytes and contribute to existing myocardium following cryo-injury of the heart in quail-to-chick proepicardial chimeras (Perez-Pomares, unpublished observations). Moreover, activated epicardial cells in adult zebrafish model of cardiac regeneration were proposed to stimulate resident cardiac progenitors within the fish heart via reciprocal Fgf-signalling [71], however, it remains an open question as to whether the activated cells in this model system have the potential to differentiate into cardiomyocytes per se.

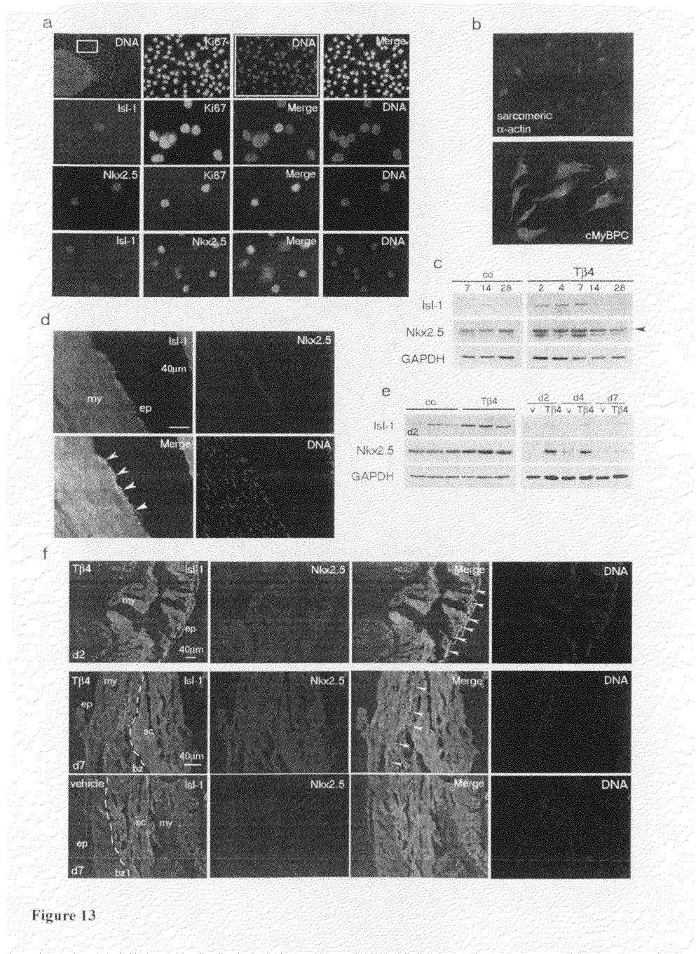

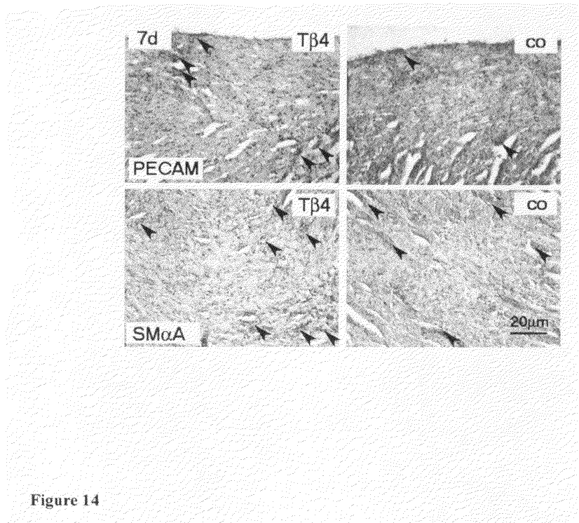

In order to determine whether Tβ4-mobilised EPDCs could differentiate into cardiomyocytes, we initially established epicardial explants as previously described¹¹ and investigated Isl1 expression as a marker of post-natal cardioblasts [14] along with recently characterised markers of embryonic cardiovascular progenitors Isl-1/Nkx2.5/Flk1 [11, 12, 13] and co-markers of progenitor proliferation (Ki-67, phospho-histone H3). Immunostaining of Tβ4 treated cultures identified proliferative cells (Ki-67 positive) emerging from the explants which were positive for both Isl-1 and Nkx2.5 (FIG. 13a) indicative of precursors of the cardiomyocyte lineage [11] and which subsequently expressed α-sarcomeric actin and α-MHC as markers of a more differentiated muscle phenotype (FIG. 13b). We next determined the presence of adult EPDC derived cardiomyocytes in vivo in both the Tβ4 gain of function and MI mouse models. In gain of function hearts, Isl-1 and Nkx2.5 were significantly up-regulated between days 2-7 of Tβ4 treatment (FIG. 13c) compared to vehicle treated control hearts and this was accompanied by robust and persistent expression of P-HH3 (FIG. 13c). In addition, Isl-1 and Nkx2.5 positive cells were readily detected by IMF in the subepicardial space following Tβ4 treatment (FIG. 13c) compared to background levels in control hearts, where indeed we failed to detect co-stained cells (data not shown). Following MI we observed significantly elevated levels of Isl1 and Nkx2.5 from 2 days post-MI onwards (FIG. 3d) and this was accompanied by an increase in Isl-1 and Nkx2.5 positive cardiac progenitors residing in the border zone of the infarct following Tβ4 treatment (FIG. 13d). Collectively this suggests that Tβ4-induced EPDCs can give rise to significantly increased levels of authentic cardioblasts above and beyond the previously identified Isl-1 positive endogenous population [14] with the potential to enter a fully differentiated cardiomyocyte lineage and regenerate injured myocardium. Evidence that these adult epicardial progenitors have an equivalent potential to their embryonic counterparts was further revealed by Flk-1 positive cells residing in the sub-epicardial space of Tβ4 treated hearts (FIG. 14).

### Modulation of Inflammation

Cardiac regeneration is intricately linked to a complex inflammatory response that must be precisely regulated to ensure proper repair and optimal cardiac outcome. Persistence of the acute inflammatory response immediately post-MI is known to extend myocardial injury (reviewed in reference 47), however, moderate inflammation is almost certainly beneficial to repair given the requirement to both remove dead or dying cardiomyocytes post injury and resolve the infarct by scar formation [47].

### Tβ4 Induces EPDC-Derived Cardiac Progenitors Ex Vivo

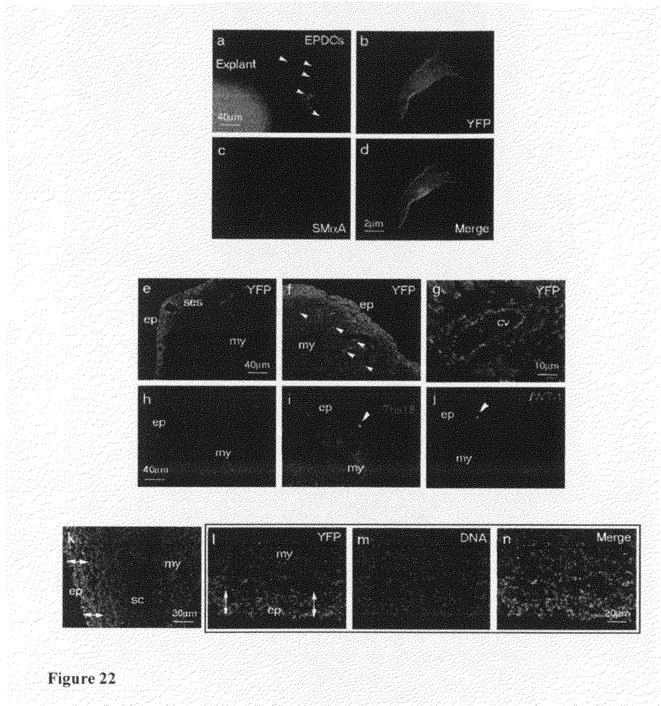

In order to determine whether Tβ4-mobilized adult EPDCs [26] could give rise to cardiomyocytes, we made use of a Gata5-EYFP epicardial trace, derived from crosses between Gata5-Cre transgenic mice [19] and a R26R-EYFP reporter strain [45]. A region of the Gata5 promoter has previously been shown to preferentially drive cre expression in the pro-epicardium and epicardial derivatives during development without effecting myocardial cells [72]. Here we demonstrate that Gata5-EYFP can act as a lineage trace for EPDCs in the adult heart both ex vivo (FIG. 22a-d), and in vivo (FIG. 22e-j). Specificity of the trace was confirmed by the fact that EYFP+ cells were not present throughout the myocardium, but restricted to a subset of myocardial cells (of embryological epicardial origin) and vascular derivatives (FIG. 22f, g). Moreover, the major population of EYFP+ cells (identified using a mouse monoclonal α-EGFP antibody which detects EYFP: α-YFP) was not only localised to the outer cell layer of the ventricles but also contained rare, isolated cells which co-stained for the fetal epicardial markers Tbx18 and WT-1 (FIG. 22h-j).

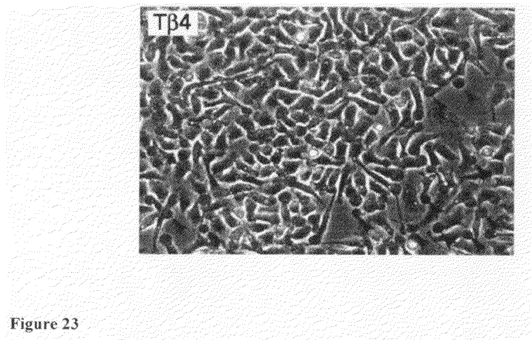

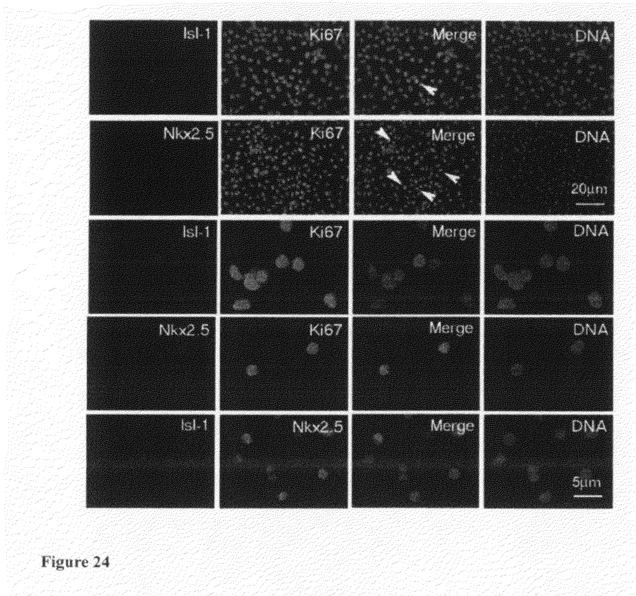

We initially established epicardial explants from adult hearts as previously described [26] and investigated Isl1 expression as a marker of post-natal cardioblasts [14] along with Nkx2.5 and Gata4, early markers of cardiomyocyte progenitors [10, 11, 12] and Ki-67, a co-marker of progenitor proliferation. At the outset we observed cells emerging from Tβ4-treated explants, up to 48 hours in culture, which appeared immature and phenotypically similar to Nkx2.5+ progenitors previously isolated from embryonic hearts [12] (FIG. 23). Immunofluorescence of Tβ4-treated cultures identified proliferative cells (Ki-67+) emerging from the explants which were positive for both Isl-1 and Nkx2.5 (FIG. 24) indicative of cardiomyocyte precursors [11].

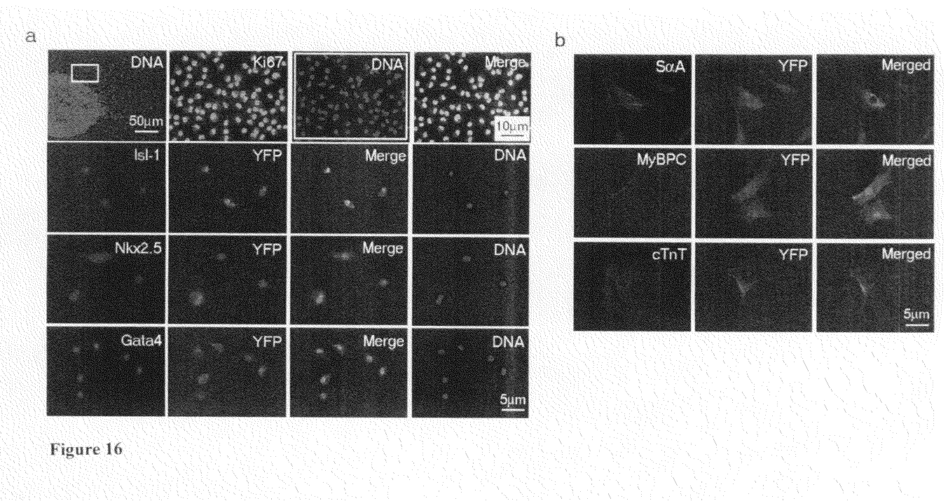

In epicardial lineage trace explants, EYFP+EPDCs were observed in cultures which co-stained for each of the early cardiac progenitor markers Isl1, Nkx2.5 and Gata4 (FIG. 16a). The percentage incidence of myocardial progenitors in the explant cultures was 5.3+/−1.9 (mean percentage of cells positive for Isl-1/Nkx2.5/Gata4+/−SEM; n=12 explants). As with non-lineage trace explants, emerging cells were all initially Ki67+ (FIG. 16a; highlighted by white box), however, with migration cells ceased to proliferate and began to differentiate into vascular precursors as previously described [26] or become committed to a myocardial lineage (FIG. 16a). Removal of the explant at 24 hours and further culture for 4 days revealed that the Isl-1/Nkx2.5/Gata4+ progenitors subsequently expressed sarcomeric α-actinin (SαA), cardiac myosin binding protein C (MyBPC) and cardiac troponin T (cTnT) as markers of a more differentiated cardiac muscle phenotype (FIG. 16b). However, none of the ex-vivo EPDC-derived cardiomyocytes exhibited sarcomeric structure suggesting that, even after 5 days in culture, the cardiomyocytes were relatively primitive and/or that the culture conditions were not optimal for cardiomyocyte terminal differentiation (FIG. 16b).

### De Novo Epicardium-Derived Cardiomyocytes are Injury-Dependent and Augmented by Tβ4

We next determined the presence of adult EPDC-derived cardiomyocytes in vivo, in both a gain of function (intact heart) mouse model, established by intraperitoneal injection of Tβ4 or vehicle (PBS) into wild type adult mice, and an injury model of myocardial infarctions (by coronary artery ligation) in adult mice (n=27 MIs in total), treated with either Tβ4

(n=13) or vehicle (n=14, injection regimen as for gain of function; refer to Methods). Hearts were assessed using a combination of western and immunofluorescence analyses for myocardial markers after 2, 4, 7, 14 and 28 days for the gain of function model and 2, 4 and 7 days for the injury model.

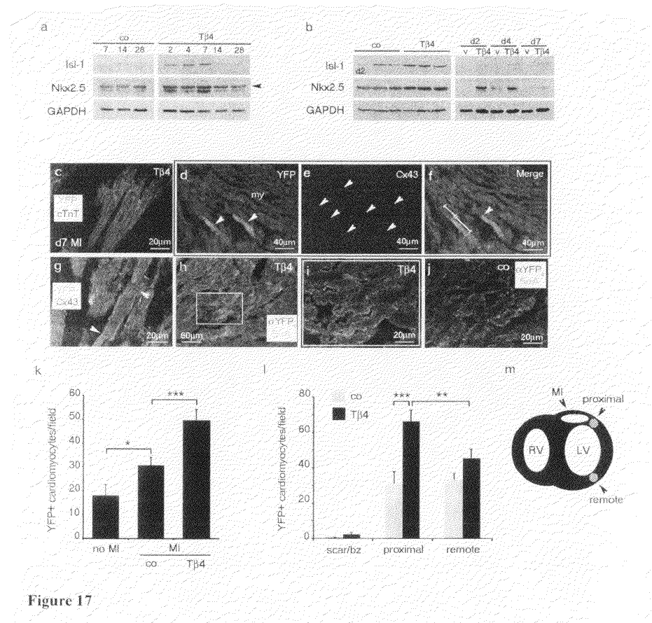

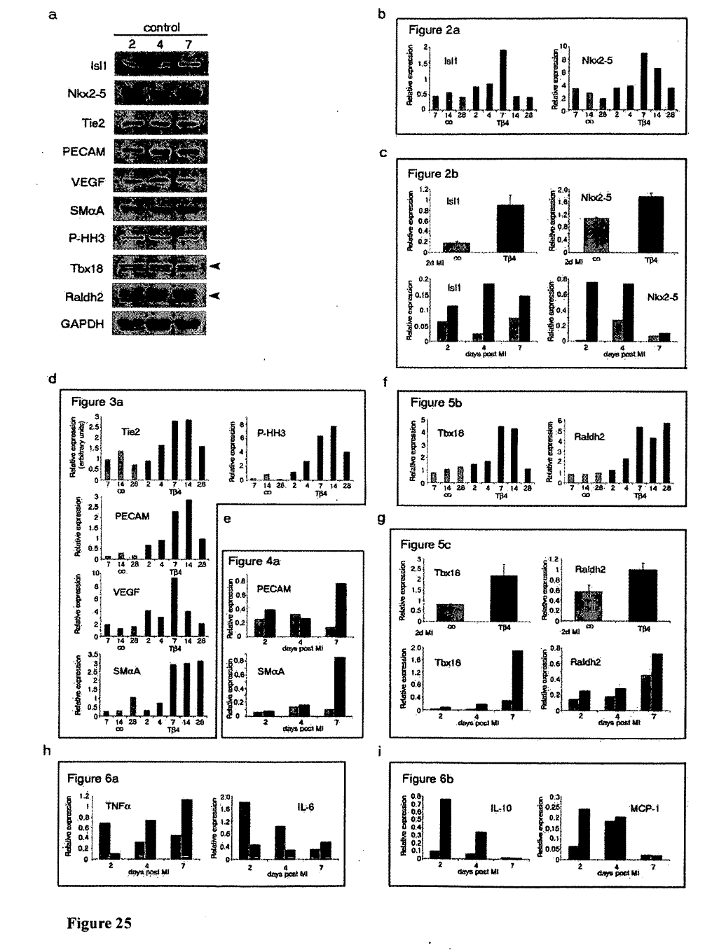

In gain of function hearts, Isl-1 (4.3-fold) and Nkx2.5 (2.7-fold) were significantly up-regulated between days 2-7 of Tβ4 treatment compared to vehicle treated control hearts (FIG. 17a; FIG. 25a, b). In control hearts there were no significant changes in expression of any of the markers investigated throughout, up to 7 days following treatment with vehicle (FIG. 25a). Scanning densitometry of western bands was used to assess quantitative changes in protein expression levels for all markers, following treatment with vehicle versus Tβ4 (FIG. 25b-i). In addition, Isl-1 and Nkx2.5 positive cells were readily detected by immunofluorescence, residing in the epicardium and adjacent subepicardial space of intact hearts, following Tβ4 treatment, compared to background levels in control hearts, where indeed we failed to detect co-stained cells (data not shown). Following MI we observed significantly elevated levels of Isl1 (5.1-fold) and Nkx2.5 (1.9-fold) from 2 days post-MI onwards (FIG. 17b; FIG. 25c); accompanied by an increase in Isl-1+ and Nkx2.5+ cardiac progenitors arising from the epicardium 2 days following Tβ4 treatment (not shown).

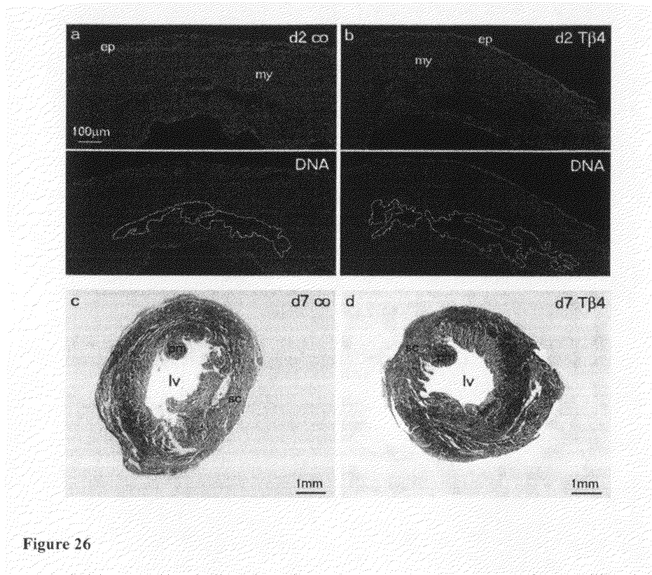

At day 7 post-MI, we observed larger EYFP+ cells, located in the wall of the left ventricle, which co-expressed cTnT, and by virtue of their size, gross morphology and inherent ultrastructure resembled mature cardiomyocytes (FIG. 17c). Importantly, these de novo cardiomyocytes were appropriately integrated with the resident myocardium as determined by connexin 43 (Cx43)+ gap junction formation (FIG. 17d-f; increased magnification in FIG. 17g). To rule out the possibility of autofluorescence accounting for the detection of the EYFP+ cardiomyocytes, we co-stained sections through the left ventricle with two polyclonal α-EGFP antibodies (both of which detect EYFP: α-YFP₁ and α-YFP₂) and either cTnT (not shown) or SαA to reveal differentiated EPDC-derived cardiomyocytes within resident myocardium (FIG. 17h-j). The specificity of both EYFP antibodies was ascertained by immunofluorescence on non-lineage trace hearts, which detected neither EPDCs nor EPDC-derived cardiomyocytes (no signal; not shown). We observed EPDC-derived cardiomyocytes in control (vehicle-treated) lineage trace hearts post-MI as either reflecting myocardial cells derived from embryological epicardium or an indicator of endogenous, albeit sub-optimal, myocardial repair (FIG. 17j). The extent of cardiomyocyte differentiation in the intact heart and during injury, with or without Tβ4 treatment, was assessed by detailed counts of cells double positive for EYFP/cTnT and α-EGFP/SαA (FIG. 17k, l). EYFP+cardiomyocytes were observed in the intact (no MI) heart indicative of an EPDC contribution to the myocardial lineage (FIG. 17k; 5.1% of total cardiomyocytes per field) and consistent with the recently reported fate of embryonic epicardial progenitors [73, 74]. We observed an increase in the number of EPDC-derived cardiomyocytes following injury in vehicle-treated hearts (8.5% of total cardiomyocytes per field) as compared to the number in the intact heart (30.79+/−3.35, vehicle post-MI v 17.86+/−4.65, intact heart; mean number of cells/field+/−SEM; n=6, total 39 fields per group, p=<0.04; FIG. 17k). This was attributed to an innate attempt to regenerate damaged muscle post-MI and was significantly enhanced by treatment with Tβ4 for 7 days (14.1% of total cardiomyocytes per field; 49.53+/−4.68 mean number of positive cells/field+/−SEM, n=6, total 38 fields, p=0.001 vs vehicle, p=0.0002 vs no MI; FIG. 17k). Since the numbers of EYFP+ cardiomyocytes increased both with injury and even more so following Tβ4-treatment this suggested de novo contribution of myocardium, as opposed to increased survival/reduced apoptosis of resident cardiomyocytes whereby the overall EYFP+ cell number would be predicted, at best, to remain static. Moreover, increased cardiomyocyte survival was unlikely to represent the exclusive mechanism of Tβ4 activity, in this context given the fact that, after 2 days post-MI, infarct size was essentially equivalent in vehicle versus Tβ4-treated hearts prior to the onset of myocardial regeneration (FIG. 26a, b); reduced scar size in the Tβ4 treated MI hearts was only apparent after 7 days (FIG. 26c, d).

EYFP+ cardiomyocytes were subsequently assessed across different regions of the left ventricle in relation to the site of injury (FIG. 171); virtually no EYFP+ cardiomyocytes were located within the scar or at the border zone; a proportion (51% of total in control, 40% of total in Tβ4-treated hearts; where “total” refers to total number of EYFP+ cardiomyocytes per field, excluding resident EYFP− cardiomyocytes) were located in the more peripheral ventricle wall (remote myocardium), and a significant number (40% of total in control, 58% of total in Tβ4 treated; p=<0.001), resided proximal to the border zone of the infarct within healthy muscle (FIG. 17l, m). This is consistent with the scar and injury border regions as inhospitable environments for cell survival (reviewed in 75), given the disturbance in extracellular matrix due to fibrosis (FIG. 26c, d) and the lack of cell-cell contact, as compared to more distal regions of intact myocardium which are permissive for integration of newly formed cardiomyocytes. A significant Tβ4-induced contribution of EYFP+ cardiomyocytes to surviving myocardium (both proximal and remote to the site of injury) may be sufficient to bring about expansion of the existing muscle mass in the LV wall, resulting in subsequent encroachment of the continuum of healthy myocardium, adjacent to the border zone, into the site of injury. Expansion of healthy myocardium into the infarct region may, therefore, contribute to the reduced scarring following Tβ4 treatment both demonstrated here (FIG. 26d) and previously reported, alongside the improvement in functional parameters, such as fractional shortening and left ventricular ejection fraction [28].

Collectively, these data suggest that EPDCs can respond to injury to contribute a basal number of de novo cardiomyocytes. Tβ4 enhances this response to induce a significant increase in EPDC-derived cardioblasts (expressing the early markers Isl1/Nkx2.5; FIG. 16a), above and beyond the previously identified Isl-1 positive endogenous population [14]. The EYFP+ myocardial progenitors subsequently adopt a terminally differentiated fate (cTnT/SαA positive myofibrils; FIG. 17c, g-j), couple to resident cardiomyocytes (via Cx43-positive gap junctions; FIG. 17e-g) and serve to regenerate injured myocardium.

### Tβ4 Induces Neovascularization in the Intact Adult Heart

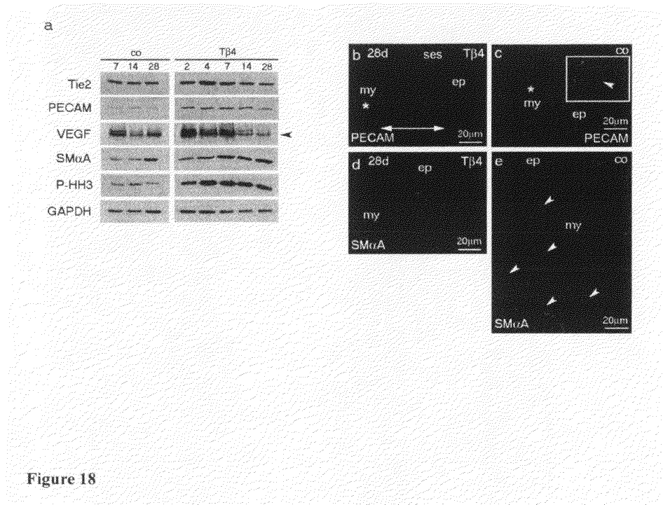

The minimum requirement for Tβ4 to promote EPDC-derived vascular endothelial and smooth muscle cells ex vivo, was reported previously [26]. To investigate whether Tβ4 can stimulate bona fide new vessel growth in vivo we first examined hearts from our gain of function model by western analysis and immunofluorescence for vascular markers and evidence of new coronary arteries at 2, 4, 7, 14 and 28 days post-treatment. Endothelial markers Tie2 (2-fold), PECAM (9.3-fold) and VEGF (4.8-fold) and the smooth muscle marker SMαA (9.8-fold) were significantly increased following 2 days of Tβ4 treatment compared to controls (FIG. 18a; FIG. 25a, d). The Tβ4-induced increase in vascular markers persisted throughout the duration of the experiment and was accompanied by a significant increase in proliferation as determined by elevated levels (9.1-fold) of phospho-histone H3 (P-HH3; FIG. 18a; FIG. 25d). Immunofluorescence on Tβ4-treated hearts showed strong regionalized staining for PECAM and SMαA, in an expanded subepicardial space and immediate underlying myocardium, with an increase in the number of coronary vessels after 28 days as indicated by endothelial cell lined arterioles (FIG. 18b, c) surrounded by smooth muscle (FIG. 18d, e). Neither expansion of the sub-epicardium nor an associated vascular network, were observed in control hearts; instead this region was entirely superficial with existing coronary vessels characteristically located deep within the underlying myocardium (FIG. 18c).

These studies suggest that not only can Tβ4 promote neovascularization in vivo but that this response can occur in the absence of injury.

### Neovascularization is Optimized in an Injury Setting

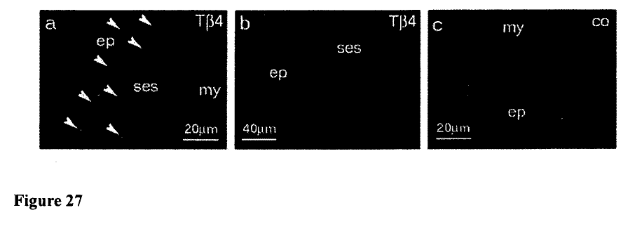

We next investigated whether Tβ4 could promote neovascularization after myocardial damage and whether this may be enhanced as compared to the intact gain of function model. Tβ4-treated, infarcted hearts had significantly increased PECAM and SMαA protein expression (9.6-fold and 8.3-fold increases respectively at d7; FIG. 19a; FIG. 25e). A significant up-regulation in vascular markers after 7 days post infarct was accompanied by an increase in cell proliferation in the epicardium and subepicardial space determined by for the presence of Ki67+ cells (FIG. 27a-c). The incidence of vascular endothelial and smooth muscle cells in the sub-epicardial space of Tβ4 treated infarcted hearts by d7 (FIG. 19b-e) was elevated beyond that observed in intact gain of function hearts at an equivalent stage (data not shown) and comparable to that observed in intact hearts after 28 days of Tβ4 treatment (compare FIG. 19b, d with FIG. 18b). Injury alone (vehicle-treated post-MI) induced an endogenous endothelial (PECAM+) response (FIG. 19c), however, there was no equivalent response at the level of smooth muscle cells (FIG. 19e). Extensive smooth muscle cell (SMαA+) migration and differentiation was only established following treatment with Tβ4 (FIG. 19d). Smooth muscle collateral growth may, therefore, explain the beneficial effects of Tβ4 treatment post-MI [28], as compared to the relatively unstable, endothelial-restricted, endogenous response (FIG. 19c).

### Tβ4-Stimulated Adult Epicardium Contributes Vascular Endothelial and Smooth Muscle Cells In Vivo

In order to assess whether Tβ4-induced coronary vessels might be epicardial in origin, we examined the incidence of PECAM+ and SMαA+ cells in Gata5-EYFP lineage trace hearts. This analysis confirmed a significant mobilisation of EPDCs following Tβ4 treatment and the presence of clusters of small proliferative EYFP+ cells, located proximal to, and in contact with, established PECAM+ and SMαA+ vessels as evidence of an ongoing contribution of EPDCs to existing vasculature (FIG. 19h, i). However, EYFP+ cell contribution was insufficient to account for the full extent of new vessel formation in the presence of Tβ4; not only were PECAM+(FIG. 19f-h) or SMαA+ (FIG. 19i) EPDCs rounded in appearance and lacking mature endothelial or smooth muscle cell morphology, but the vascular plexus formed in the subepicardial space was devoid of EYFP+ cells (not shown). The lack of direct adult EPDC contribution to the mature endothelial lineage in vivo is consistent with the restricted potential (smooth muscle, fibroblast, myocardial cells) recently attributed to embryological EPDCs in the developing heart [74]. BrdU labelling revealed an absence of proliferating PECAM+ cells at the site of native vessels (data not shown), suggesting vascular expansion by Tβ4 was due, at least in part, to de novo vasculogenesis, consistent with that observed in the expanded subepicardial region (FIG. 18b).

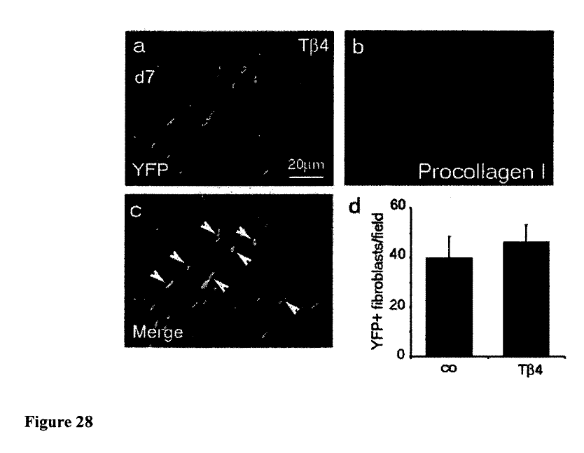

In lineage trace, control hearts, EPDCs were observed to mobilize from the epicardium post-MI and migrate into the underlying ventricular myocardium (FIG. 22i-l) as evidence of an endogenous, albeit inefficient, EPDC-response to injury. In addition, we also detected EYFP+ cells co-stained with procollagen type I as a marker for fibroblasts (FIG. 28a-c; including fibroblast cell numbers: FIG. 28d) consistent with our previous observations, in Tβ4-stimulated explant cultures, of a restoration of embryonic pluripotency to activated adult EPDCs [26].

Collectively these findings suggested optimal Tβ4-induced neovascularization in the injury setting, and were confirmed by a quantitative assessment of Tβ4-induced coronary vasculature for both the gain of function and injury models. Counts of rhodamine-dextran-perfused vessels in Connexin40 (Cx40)-EGFP transgenic mice, which labels all coronary arteries [46], revealed a significant 1.2-fold increase in numbers of perfused coronary vessels (smooth muscle-lined arterioles) following 28 days of Tβ4 treatment, compared to controls (70.7+/−3.62, Tβ4 v 57.4+/−2.97, control (co); number of perfused vessels per section+/−SEM, n=3; 60 fields imaged at 5 separate comparable levels through the heart, p=0.008; FIG. 19j). An assessment of PECAM+ cells in infarcted hearts revealed a highly significant, 3.5-fold increase in the number of endothelial cells in Tβ4 versus vehicle treated after only 4 days (150.33+/−10.94, Tβ4 v 42.25+/−5.17, control; n=3; number of cells per field+/−SEM, p=9.09×10⁻⁹; FIG. 19k). Image J analyses further revealed a significant 2-fold increase in vessel area after 7 days of Tβ4 treatment post-MI (10.67+/−0.55% vessel area/field, Tβ4 v 6.69+/−0.60%, co; n=7; 6 fields per heart at comparable levels; p=9.45×10⁻⁶; FIG. 19l). Moreover, in support of the observed injury-induced endogenous neovascularization, the overall increase in vessel/arteriole area in vehicle-treated hearts was dependent upon the severity of injury (4.75% in mild injury v. 11.38% in severe injury; % vessel area/field) measured as infarct area/total LV area (mean score of infarct area over 10 sections at comparable levels; FIG. 19m).

In conclusion, Tβ4 initiated a significant vascular response in the intact heart, which was further enhanced following injury to give rise to de novo functional (perfused) vessels in vivo; thus Tβ4 acts synergistically with injury-induced vasculogenic signalling. EPDCs are activated as an endogenous response to injury but although they are observed to contribute vascular “progenitors” to new or existing coronary vessels (FIG. 19h, i) they do not account for the full extent of Tβ4-induced neovascularization.

### Tβ4 Induces the Adult Epicardium Via Reactivation of Fetal Genes

Unlike in adult mammals, where the heart is one of the least regenerative organs in the body [76], the adult zebrafish has retained the capacity for cardiac regeneration [77]. Repair of the injured heart in the zebrafish is underpinned by organ-wide activation of the epicardium which retains or re-expresses embryonic epicardial markers [71]. Induction of the so-called fetal gene program also precedes and accompanies cardiac hypertrophy, as an intrinsic adaptive response of the heart to pathological signalling, which involves changes in cellular phenotype [78].

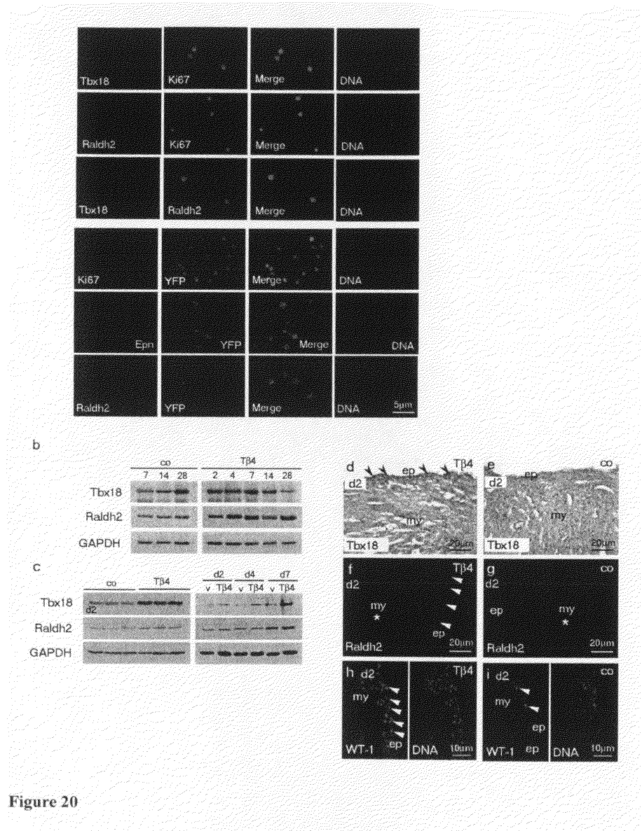

Therefore, we sought to investigate whether Tβ4 treatment could bring about reactivation of key genes such as Tbx18, Raldh2, Epicardin and Wt-1, which are preferentially expressed in the developing embryonic epicardium, as an indicator of quiescent adult epicardial cells adopting an embryonic multipotent fate. Tβ4 addition to epicardial explants resulted in a significant number of proliferative, migrating EPDCs positive for Tbx18 and Raldh2 as determined by immunofluorescence (FIG. 20a). Elevated cellular proliferation and enhanced migratory capacity, as induced by Tβ4 within the adult epicardium, is entirely consistent with the phenotype of embryonic EPDCs as they undergo epithelial-mesenchyme transition (EMT), migrate across the subepicardial space, and give rise to the cellular components of the coronary blood vessels. During development, this remodelling process is accompanied by epicardial expression of genes such as Tbx18 and Raldh2 which serve as surrogate markers of activated embryonic EPDCs. Furthermore, in Gata5-EYFP lineage traced explants (FIG. 22a-d) we observed EYFP positive EPDCs which were proliferative as determined by co-staining for Ki67 and which expressed Tbx18, Raldh2 and the epicardial transcription factor Epicardin following stimulation by Tβ4 (FIG. 20a). Equally, in both the in vivo gain of function and MI models, Tβ4 addition invoked a significant up-regulation of Tbx18 and Raldh2 as compared to vehicle treated control hearts (Tbx18: 5.3-fold and 2.8-fold; Raldh2: 6.3-fold and 1.8-fold in gain of function and d2 MI respectively; FIG. 20b, c; FIG. 25f, g). In the untreated, injured heart Tbx18 and Raldh2 were only expressed at low levels, however, in response to Tβ4, both proteins were up-regulated in the treated adult epicardium (FIG. 20b, c; FIG. 25f, g). Immunohistochemistry and immunofluorescence on infarcted hearts, after 2 days of Tβ4 treatment, confirmed Tbx18 (FIG. 20d, e), Raldh2 (FIG. 20f, g) and additionally WT-1 (FIG. 20 h, i) localization to the adult epicardium. Specifically these markers were expressed in delaminating EPDCs and those migrating through the subepicardial space into the underlying myocardium; highly reminiscent of embryonic heart development (FIG. 20d-i). Tbx18+ and Raldh2+ cells were detected in vehicle-treated hearts post-infarction, consistent with a moderate activation of EPDCs in response to injury; EYFP+ epicardial cells rarely co-expressed embryonic genes such as Tbx18 or WT-1 in the intact heart (FIG. 22i, j). The injury response was significantly increased following treatment with Tβ4 (FIG. 20d-g; Tbx18: 14.3+/−2.3, in control vs 36.1+/−3.1 in Tβ4-treated, p=<0.05; Raldh2: 9.1+/−2.2 in control vs 41.8+/−1.9 in Tβ4-treated, p=<0.01; mean number of positive cells per field+/−SEM; n=3; total 35 fields per group). Rarely were isolated cells expressing WT-1 identified in the controls (FIG. 20i).

Thus the mechanism of Tβ4 activation of quiescent adult epicardium appears to involve “reprogramming” epicardial cells to an embryological progenitor cell fate, whereby they can proliferate, migrate and give rise to vascular and myocardial precursors.

Acute pro- and Anti-Inflammatory Cytokines are Altered Following Tβ4 Treatment Post-Injury

Post-infarction cardiac regeneration is regulated through timely activation and repression of inflammatory pathways. Persistence of the acute inflammatory response immediately post-MI is known to extend myocardial injury (reviewed in 47), however, moderate inflammation is almost certainly beneficial to repair, given the requirement to both remove dead or dying cardiomyocytes post injury and resolve the infarct with granulation tissue [47].

There is a growing weight of evidence to suggest that Tβ4 can exert both anti-inflammatory and anti-fibrotic effects. In mammals (including humans) MI tends to result in persistent acute inflammation and scarring which contributes significantly to impaired cardiac performance, therefore, we investigated whether Tβ4 might regulate inflammation post-MI in the adult mouse heart.

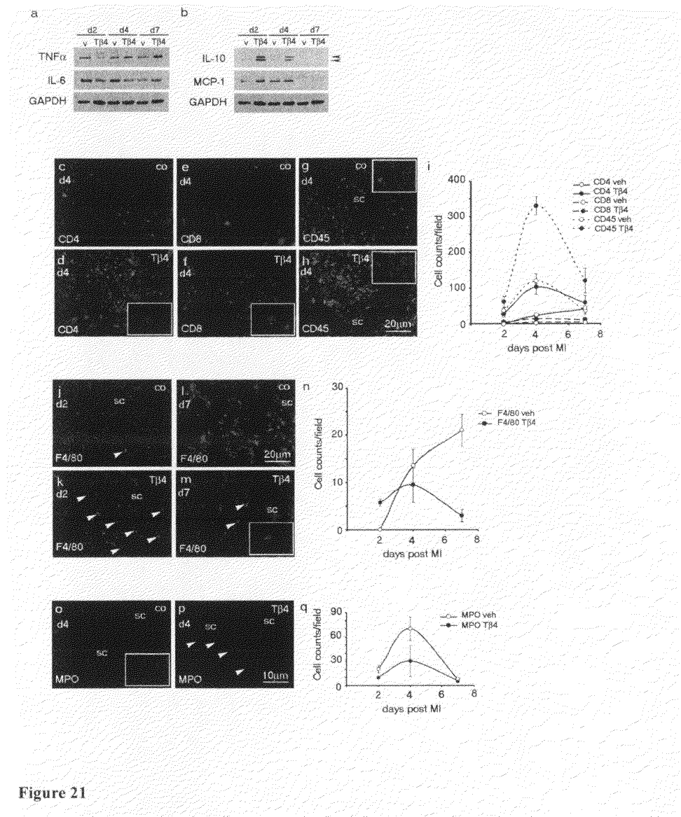

In the first instance we investigated the effect of Tβ4 treatment on the levels of mast cell-derived TNF-α, as the upstream cytokine responsible for initiating the inflammatory cascade [90, 91]. TNF-α was significantly reduced (6.2-fold reduction) after 2 days of Tβ4 treatment post-MI as compared to vehicle-treated controls (FIG. 21a; FIG. 25h). The early effects on TNF-α suggest that Tβ4 may act to inhibit the initial inflammatory response post-myocardial injury. Consistent with this, treatment of Tβ4 also resulted in significantly reduced levels of downstream factors such as the proinflammatory cytokine IL-6 (4.1-fold reduction), which is rapidly induced in the ischemic myocardium to mediate neutrophil-induced injury (FIG. 21a; FIG. 25h). Importantly, although reduced at early stages post-MI (d2 and d4), components of the TNF-α inflammatory cascade were all observed to rise again by d7 following Tβ4 treatment indicative of Tβ4 inhibiting the early acute inflammatory response known to enhance injury but promoting later stage cardiac repair (FIG. 21a; FIG. 25h).

### Immune Cell Infiltration is Reduced by Tβ4 Immediately Post-Injury But Restored to Promote Cardiac Repair

Further evidence that Tβ4 stimulates cardiac repair, at the expense of inflammatory-induced injury, arose from an observed early up-regulation of the potent monocyte chemoattractant protein MCP-1 (3.9-fold) in Tβ4 treated infarcted hearts (FIG. 21b; FIG. 25i). Moreover, elevated MCP-1, at d2 post-MI, was accompanied by infiltration of the border zone with T lymphocytes and monocyte macrophages (FIG. 21c-i and 21j-n) which act to clear necrotic debris and enable more effective healing [47]. This response, which typically occurs 48-72 hours post-infarct [79], was induced more rapidly and to a greater extent in Tβ4-treated MI hearts compared with vehicle, as determined by numbers of CD4+ (T helper) versus CD8+ (cytotoxic T cells) and CD45+ (pan leukocytes) cells (FIG. 21i) and F4/80+ (activated) macrophages (FIG. 21n). Consistent with this early beneficial effect of Tβ4 treatment we observed a reduced infiltration of MPO+ neutrophils in Tβ4-treated hearts compared to controls at d2 and d4 post-MI (FIG. 21o-q); trapping of neutrophils during ischaemic injury is known to enhance pathophysiology and prevent reperfusion of capillaries [80, 81].

Delayed clearance of the post-MI immune cell response is associated with augmented myocardial injury (reviewed in 47). The Tβ4-induced monocyte-rich infiltrate was efficiently cleared by d7 post-MI (FIGS. 21i and n), in addition, Tβ4 treatment resulted in an elevated CD4:CD8 ratio compared with vehicle at both d4 (CD4:CD8: 7.4, Tβ4 and 4.5, co) and d7 (CD4:CD8: 10.8, Tβ4 and 8.3, co) post-MI (FIG. 21i). A relative increase in T-helper versus cytotoxic T-cells is key to improved clinical prognosis in patients with acute MI, since an inverted ratio of CD4/CD8 cells is associated with poor outcome [82].

In keeping with a role in mitigating inflammatory injury during the early stages post-infarct without interfering with subsequent myocardial healing, Tβ4 treatment stimulated the expression of the inhibitory cytokine IL-10 at 2 days post-MI (8.1-fold increase compared to control; FIG. 21b; FIG. 25i). IL-10 is thought to suppress the acute inflammatory response and contribute extensively to effective repair via regulating extracellular matrix (ECM) metabolism. Although the precise mechanism underlying the role of Tβ4 in modulating inflammation and wound healing requires further analysis, these data are consistent with the reported ability of Tβ4 to reduce fibrosis and scarring post-MI (FIG. 26).

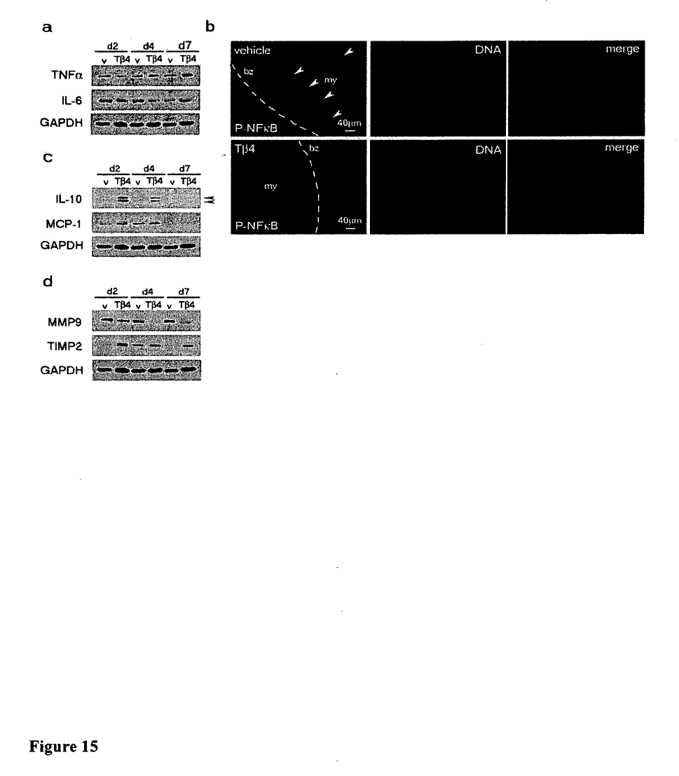

Regeneration and inflammation/fibrosis are competing events in the vertebrate heart and the latter exists as a default pathway even in the adult zebrafish despite its high cardiac regenerative capacity [1]. This suggests that injury-stimulated cardiomyocyte hyperplasia beyond a certain threshold in the fish ensures regenerative mechanisms can overcome scarring [1]. Tβ4 is up-regulated during zebrafish cardiac regeneration [83,] and there is a growing weight of evidence to suggest that Tβ4 can exert both anti-inflammatory and anti-fibrotic [84 to 89] effects. In mammals (including humans) MI tends to result in persistent acute inflammation and scarring which contributes significantly to impaired cardiac performance [47], therefore, we investigated whether Tβ4 can regulate inflammation and fibrosis post-MI in the adult mouse heart. In the first instance we investigated the effect of Tβ4 treatment on the levels of mast cell derived TNF-α, as the upstream cytokine responsible for initiating the inflammatory cascade [90, 91]. TNF-α was significantly reduced after 2 days of Tβ4 treatment post-MI as compared to vehicle controls (FIG. 15a). Since TNFα initiates signalling pathways that converge on the activation of NFkB to mediate inflammation [47] we subsequently examined a putative role for Tβ4 in modulating NFκB activity in the infarcted heart by assessment of NFκB phosphorylation status and immunostaining on sections through the infarct region with phospho-specific (phosphoserine 276 p65) and pan-NFκB specific (non phosphorylated p65) antibodies. Treatment of injured hearts with Tβ4 brought about a reduction in globally activated NFκB expression in whole hearts and a reduced number of P—NFκB positive cells at the infarct border zone (FIGS. 15a and b). The early effects on the TNF-α/NFκB pathway suggest that Tβ4 may act to inhibit the early inflammatory response post-myocardial injury. Consistent with this, treatment of Tβ4 also resulted in significantly reduced levels of downstream factors such as the proinflammatory cytokine IL-6, which is rapidly induced in the ischaemic myocardium to mediate neutrophil-induced injury, and the chemoattractant protein MCP-1 which induces recruitment of mononuclear cells during the initial stages post-MI (FIG. 5c) Furthermore, in keeping with a role in mitigating inflammatory injury during the early stages post-infarct without interfering with subsequent myocardial healing, Tβ4 treatment stimulated the expression of the inhibitory cytokine IL-10 at 2 and 4 days post-MI (FIG. 15c). IL-10 is thought to suppress the acute inflammatory response and contribute extensively to effective repair via regulating extracellular matrix (ECM) metabolism [47]. In order to investigate an effect on the ECM we examined expression of matrix metalloproteinases (MMPs) and their associated tissue inhibitors (TIMPs). Specifically we focused on MMP3 and -9 and TIMP1 since in transgenic mice with cardiac over-expression of TNFα these factors were all elevated and associated with accelerated development of ECM remodelling and development of decompensated heart failure [92]. In Tβ4 treated hearts post MI we observed reduced levels of MMP3 and -9 without an effect on associated levels of TIMP1 and -2 (FIG. 5d) thus modulating the MMP/TIMP balance in favour of matrix repair and maintaining basement membrane integrity as an essential step in myocardial wound healing.

The adult fish model of cardiac regeneration is based on an inherent ability to mobilise epicardial cells to cultivate what is described as a vascularised “niche” and cardiogenic environment [71]. In the absence of external stimulus mammalian hearts typically show insufficient neovascularisation and consequently no myocardial regeneration after infarction. Here we identify Tβ4 as the external stimulus for mammalian cardiac regeneration, mediated by adult EPDCs which are mobilised as bona fide cardiovascular progenitors. Moreover, Tβ4 acts an anti-inflammatory agent which when combined with EPDC cultivation of new vasculature and muscle growth acts to tip the balance in favour of regeneration over scarring in the adult mammalian heart. The application of Tβ4-stimulated EPDCs facilitating survival, recovery and regenerative replacement of destroyed myocardium is a significant step towards therapy for acute MI in humans.

### REFERENCES

[1] Poss, K. D. Getting to the heart of regeneration in zebrafish. Semin. Cell Dev. Biol. 18, 36-45 (2007).
[2] Cavasin, M. A. Therapeutic Potential of Thymosin-beta4 and its Derivative N-acetyl-seryl-aspartyl-lysyl-proline (Ac-SDKP) in Cardiac Healing After Infarction. Am. J. Cardiovasc. Drugs 6, 305-311 (2006).
[3] Tomanek, R., Zheng, W. & Yue, X. Growth factor activation in myocardial vascularization: Therapeutic implications. Mol. Cell. Biochem. 264, 3-11 (2004).
[4] Orlic, D. et al. Bone marrow cells regenerate infarcted myocardium. Nature 410, 701-705 (2001).
[5] Beltrami, A. P. et al. Adult cardiac stem cells are multipotent and support myocardial regeneration. Cell 114, 763-776 (2003).
[6] Anversa, P. & Nadal-Ginard, B. Myocyte renewal and ventricular remodelling. Nature 415, 240-243 (2002).
[7] Luttun, A. & Carmeliet, P. De novo vasculogenesis in the heart. Cardiovasc. Res. 58, 378-389 (2003).
[8] Kamihata, H. et al. Implantation of bone marrow mononuclear cells into ischemic myocardium enhances collateral perfusion and regional function via side supply of angioblasts, angiogenic ligands, and cytokines. Circulation 104, 1046-1052 (2001).
[9] Saiura, A., Sata, M., Hirata, Y., Nagai, R. & Makuuchi, M. Circulating smooth muscle progenitor cells contribute to atherosclerosis. Nature Med. 7, 382-383 (2001).
[10] Srivastava, D. & Ivey, K. N. Potential of stem-cell-based therapies for heart disease. Nature 441, 1097-1099 (2006).
[11] Moretti, A. et al. Multipotent Embryonic Isl1(+) Progenitor Cells Lead to Cardiac, Smooth Muscle, and Endothelial Cell Diversification. Cell (2006).
[12] Wu, S. M. et al. Developmental Origin of a Bipotential Myocardial and Smooth Muscle Cell Precursor in the Mammalian Heart. Cell (2006).
[13] Prall, O. W. et al. An nkx2-5/bmp2/smad1 negative feedback loop controls heart progenitor specification and proliferation. Cell 128, 947-959 (2007).
[14] Laugwitz, K. L. et al. Postnatal isl1+ cardioblasts enter fully differentiated cardiomyocyte lineages. Nature 433, 647-653 (2005).
[15] Beltrami A P, Barlucchi L, Torella D, Baker M, Limana F, Chimenti S, Kasahara H, Rota M, Musso E, Urbanek K, Leri A, Kajstura J, Nadal-Ginard B, Anversa P. Adult cardiac stem cells are multipotent and support myocardial regeneration. Cell. 2003 Sep. 19; 114(6):763-76.
[16] Bearzi C, Rota M, Hosoda T, Tillmanns J, Nascimbene A, De Angelis A, Yasuzawa-Amano S, Trofimova I, Siggins R W, Lecapitaine N, Cascapera S, Beltrami A P, D'Alessandro D A, Zias E, Quaini F, Urbanek K, Michler R E, Bolli R, Kajstura J, Leri A, Anversa P. Human cardiac stem cells. Proc Natl Acad Sci USA. 2007 Aug. 28; 104(35):14068-73.
17 Kraus, F., Haenig, B. & Kispert, A. Cloning and expressing analysis of the mouse T-box gene Tbx18. Mech. Dev. 100, 83-86 (2001)
18 Moss, J. B. et al. Dynamic patterns of retinoic acid synthesis and response in the developing mammalian heart. Dev. Biol. 199, 55-71 (1998)
[19] Merki E, Zamora M, Raya A, Kawakami Y, Wang J, Zhang X, Burch J, Kubalak S W, Kaliman P, Belmonte J C, Chien K R, Ruiz-Lozano P. Epicardial retinoid X receptor alpha is required for myocardial growth and coronary artery formation. Proc Natl Acad Sci USA. 2005 Dec. 20; 102(51):18455-60.
[20] Robb et al., Epicardin: A novel basic helix-loop-helix transcription factor gene expressed in epicardium, branchial arch myoblasts, and mesenchyme of developing lung, gut, kidney, and gonads. Dev Dyn. 213(1):105-13 (1998)
21 Perez-Pomares J M, Phelps A, Sedmerova M, Carmona R, Gonzalez-Iriarte M, Munoz-Chapuli R, Wessels A. Experimental studies on the spatiotemporal expression of WT1 and RALDH2 in the embryonic avian heart: a model for the regulation of myocardial and valvuloseptal development by epicardially derived cells (EPDCs). Dev Biol. 2002 Jul. 15; 247(2):307-26.
22 Compton L A, Potash D A, Mundell N A, Barnett J V. Transforming growth factor-beta induces loss of epithelial character and smooth muscle cell differentiation in epicardial cells. Dev Dyn. 2006 January; 235(1):82-93.
[23] Gerdes et al., Cell cycle analysis of a cell proliferation-associated human nuclear antigen defined by the monoclonal antibody Ki-67. J Immunol 133: 1710-1715 (1984)
[24] Hendzel et al., Mitosis-specific phosphorylation of histone H3 initiates primarily within pericentromeric heterochromatin during G2 and spreads in an ordered fashion coincident with mitotic chromosome condensation. Chromosoma. 106(6):348-60 (1997)
[25] Srinivas et al., Cre reporter strains produced by targeted insertion of EYFP and ECFP into the ROSA26 locus. BMC Dev Biol.; 1:4 (2001)
[26] Smart et al., Thymosin β4 induces adult epicardial progenitor mobilization and neovascularisation. Nature 445: 177-182 (2007)
[27] Thomas, D. L., Lythgoe, M. F., Gadian, D. G. & Ordidge, R. J. In vivo measurement of the longitudinal relaxation time of arterial blood (T1a) in the mouse using a pulsed arterial spin labeling approach. Magn Reson. Med. 55, 943-947 (2006).
[28] Bock-Marquette, I., Saxena, A., White, M. D., Dimaio, J. M. & Srivastava, D. Thymosin beta4 activates integrin-linked kinase and promotes cardiac cell migration, survival and cardiac repair. Nature 432, 466-472 (2004).
[29] Grant et al., Matrigel induces thymosin beta 4 gene in differentiating endothelial cells. J Cell Sci.; 108:3685-94 (1995)
[30] Moses, K., DeMayo, F., Braun, R., Reecy, J. & Schwartz, R. Embryonic expression of an Nkx2-5/Cre gene using ROSA26 reporter mice. Genesis 31, 176-180 (2001).
[31] Zeisberg, E. M. et al. Morphogenesis of the right ventricle requires myocardial expression of Gata4. J. Clin. Invest. 115, 1522-1531 (2005).
[32] Chen, J. et al. Selective requirement of myosin light chain 2v in embryonic heart function. J. Biol. Chem. 273, 1252-1256 (1998).
[33] Wiesmann, F. et al. Developmental changes of cardiac function and mass assessed with MRI in neonatal, juvenile, and adult mice. Am. J. Physiol Heart Circ. Physiol 278, H652-H657 (2000).
[34] Wiesmann, F. et al. Analysis of right ventricular function in healthy mice and a murine model of heart failure by in vivo MRI. Am. J. Physiol Heart Circ. Physiol 283, H1065-H1071 (2002).
[35] De Souza, A. T. et al. Transcriptional and phenotypic comparisons of Ppara knockout and siRNA knockdown mice. Nucleic Acids Res. 34, 4486-4494 (2006).
[36] Zhou, R., Pickup, S., Glickson, J. D., Scott, C. H. & Ferrari, V. A. Assessment of global and regional myocardial function in the mouse using cine and tagged MRI. Magn Reson. Med. 49, 760-764 (2003).
[37] Kober, F., lltis, I., Cozzone, P. J. & Bernard, M. Myocardial blood flow mapping in mice using high resolution spin labeling magnetic resonance imaging: influence of ketamine/xylazine and isoflurane anesthesia. Magn Reson. Med. 53, 601-606 (2005).
[38] Fernandez, B. et al. Transgenic myocardial overexpression of fibroblast growth factor-1 increases coronary artery density and branching. Circ. Res. 87, 207-213 (2000).
[39] Nahrendorf, M. et al. Chronic coronary artery stenosis induces impaired function of remote myocardium: MRI and spectroscopy study in rat. Am. J. Physiol Heart Circ. Physiol 285, H2712-H2721 (2003).
[40] Bai et al., Genetically selected stem cells from human adipose tissue express cardiac markers. Biochem Biophys Res Commun. 16; 353(3):665-71 (2007)
[41] Gissel et al., Generation and characterization of cardiomyocytes under serum-free conditions. Methods Mol. Biol. 330:191-219 (2006)
[42] Apkon and Nerebonne, Characterization of two distinct depolarization-activated K+ currents in isolated adult rat ventricular myocytes. 97(5):973-1011 (1991)
[43] Thomas, D. L., Lythgoe, M. F., Gadian, D. G. & Ordidge, R. J. In vivo measurement of the longitudinal relaxation time of arterial blood (T1a) in the mouse using a pulsed arterial spin labeling approach. Magn Reson. Med. 55, 943-947 (2006).
[44] Merki, E. et al. Epicardial retinoid X receptor alpha is required for myocardial growth and coronary artery formation. Proc. Natl. Acad. Sci. U.S. A 102, 18455-18460 (2005).
[45] Srinivas, S. et al. Cre reporter strains produced by targeted insertion of EYFP and ECFP into the ROSA26 locus. BMC. Dev. Biol. 1, 4 (2001).
[46] Miquerol, L. et al. Architectural and functional asymmetry of the His-Purkinje system of the murine heart. Cardiovasc. Res. 63, 77-86 (2004).
[47] Frangogiannis, N. G., Smith, C. W. & Entman, M. L. The inflammatory response in myocardial infarction. Cardiovasc. Res. 53, 31-47 (2002).
[48] Fijnvandraat, A. C., De Boer, P. A., Deprez, R. H. & Moorman, A. F. Nonradioactive in situ detection of mRNA in ES cell-derived cardiomyocytes and in the developing heart. Microsc. Res. Tech. 58, 387-394 (2002).
[49] Moses, K., DeMayo, F., Braun, R., Reecy, J. & Schwartz, R. Embryonic expression of an Nkx2-5/Cre gene using ROSA26 reporter mice. Genesis 31, 176-180 (2001).
[50] Zeisberg, E. M. et al. Morphogenesis of the right ventricle requires myocardial expression of Gata4. J. Clin. Invest. 115, 1522-1531 (2005).
[51] Chen, J. et al. Selective requirement of myosin light chain 2v in embryonic heart function. J. Biol. Chem. 273, 1252-1256 (1998).
[52] Ward, N. L. & Dumont, D. J. The angiopoietins and Tie2/Tek: adding to the complexity of cardiovascular development. Semin. Cell Dev. Biol. 13, 19-27 (2002).
[53] von Kodolitsch, Y. et al. Coronary artery anomalies Part I: Recent insights from molecular embryology. Z. Kardiol. 93, 929-937 (2004).
[54] Merki, E. et al. Epicardial retinoid X receptor a is required for myocardial growth and coronary artery formation. Proc. Natl Acad. Sci. USA 102, 18455-18460 (2005).
[55] Yamashita, J. et al. Flk1-positive cells derived from embryonic stem cells serve as vascular progenitors. Nature 408, 92-96 (2000).
[56] Carmeliet, P. Angiogenesis in health and disease. Nature Med. 9, 653-660 (2003).
[57] Tevosian, S. G. et al. FOG-2, a cofactor for GATA transcription factors, is essential for heart morphogenesis and development of coronary vessels from epicardium. Cell 101, 729-739 (2000).
[58] Giordano, F. J. et al. A cardiac myocyte vascular endothelial growth factor paracrine pathway is required to maintain cardiac function. Proc. Natl. Acad. Sci. USA 98, 5780-5785 (2001).
[59] Morabito, C. J., Dettman, R. W., Kattan, J., Collier, J. M. & Bristow, J. Positive and negative regulation of epicardial-mesenchymal transformation during avian heart development. Dev. Biol. 234, 204-215 (2001).
[60] Chen, T. H. et al. Epicardial induction of fetal cardiomyocyte proliferation via a retinoic acid-inducible trophic factor. Dev. Biol. 250, 198-207 (2002).
[61] Poelmann, R. E., Lie-Venema, H. & Gittenberger-de Groot, A. C. The role of the epicardium and neural crest as extracardiac contributors to coronary vascular development. Tex. Heart Inst. J. 29, 255-261 (2002).
[62] Luttun, A. & Carmeliet, P. De novo vasculogenesis in the heart. Cardiovasc. Res. 58, 378-389 (2003).
[63] Kamihata, H. et al. Implantation of bone marrow mononuclear cells into ischemic myocardium enhances collateral perfusion and regional function via side supply of angioblasts, angiogenic ligands, and cytokines. Circulation 104, 1046-1052 (2001).
[64] Saiura, A., Sata, M., Hirata, Y., Nagai, R. & Makuuchi, M. Circulating smooth muscle progenitor cells contribute to atherosclerosis. Nature Med. 7, 382-383 (2001).
[65] Grillon, C. et al. Involvement of thymosin β4 and endoproteinase Asp-N in the biosynthesis of the tetrapeptide AcSerAspLysPro a regulator of the hematopoietic system. FEBS Lett. 274, 30-34 (1990).
[66] Rieger, K. J. et al. Involvement of human plasma angiotensin 1-converting enzyme in the degradation of the haemoregulatory peptide N-acetyl-seryl-aspartyl-lysylproline. Biochem. J. 296, 373-378 (1993).
[67] Goldstein, A. L., Hannappel, E. & Kleinman, H. K. Thymosin β4: actin-sequestering protein moonlights to repair injured tissues. Trends Mol. Med. II, 421-429 (2005).
[68] Wang, D. et al. N-acetyl-seryl-aspartyl-lysyl-proline stimulates angiogenesis in vitro and in vivo. Am. J. Physiol. Heart Circ. Physiol. 287, H2099-H2105 (2004).
[69] Pokharel, S. et al. Increased myocardial collagen content in transgenic rats overexpressing cardiac angiotensin-converting enzyme is related to enhanced breakdown of N-acetyl-ser-asp-lys-pro and increased phosphorylation of Smad2/3. Circulation 110, 3129-3135 (2004).
[70] Peng, H. et al. Angiotensin-converting enzyme inhibitors: A new mechanism of action. Circulation 112, 2436-2445 (2005).
[71] Lepilina, A. et al. A Dynamic Epicardial Injury Response Supports Progenitor Cell Activity during Zebrafish Heart Regeneration. Cell 127, 607-619 (2006).
[72] Zamora, M., Manner, J., & Ruiz-Lozano, P. Epicardium-derived progenitor cells require beta-catenin for coronary artery formation. Proc. Natl. Acad. Sci. U.S. A 104, 18109-18114 (2007).
[73] Zhou, B. et al. Epicardial progenitors contribute to the cardiomyocyte lineage in the developing heart. Nature (2008).
[74] Cai, C. L. et al. A myocardial lineage derives from Tbx18 epicardial cells. Nature (2008).
[75] Frangogiannis, N. G. The mechanistic basis of infarct healing. Antioxid. Redox. Signal. 8, 1907-1939 (2006).
[76] Laflamme, M. A. & Murry, C. E. Regenerating the heart. Nat. Biotechnol. 23, 845-856 (2005).
[77] Poss, K. D., Wilson, L. G., & Keating, M. T. Heart regeneration in zebrafish. Science 298, 2188-2190 (2002).
[78] Frey, N. & Olson, E. N. Cardiac hypertrophy: the good, the bad, and the ugly. Annu. Rev. Physiol 65, 45-79 (2003).
[79] Liao, Y. H. & Cheng, X. Autoimmunity in myocardial infarction. Int. J. Cardiol. 112, 21-26 (2006).
[80] Jolly, S. R. et al. Reduction of myocardial infarct size by neutrophil depletion: effect of duration of occlusion. Am. Heart J. 112, 682-690 (1986).
[81] Litt, M. R., Jeremy, R. W., Weisman, H. F., Winkelstein, J. A., & Becker, L. C. Neutrophil depletion limited to reperfusion reduces myocardial infarct size after 90 minutes of ischemia. Evidence for neutrophil-mediated reperfusion injury. Circulation 80, 1816-1827 (1989).
[82] Syrjala, H., Surcel, H. M., & Ilonen, J. Low CD4/CD8 T lymphocyte ratio in acute myocardial infarction. Clin. Exp. Immunol. 83, 326-328 (1991).
[83] Lien, C. L., Schebesta, M., Makino, S., Weber, G. J. & Keating, M. T. Gene Expression Analysis of Zebrafish Heart Regeneration. PLoS. Biol. 4, (2006).
[84] Sosne, G. et al. Thymosin beta 4 promotes corneal wound healing and decreases inflammation in vivo following alkali injury. Exp. Eye Res. 74, 293-299 (2002).
[85] Sosne, G., Christopherson, P. L., Barrett, R. P. & Fridman, R. Thymosin-beta4 modulates corneal matrix metalloproteinase levels and polymorphonuclear cell infiltration after alkali injury. Invest Opthalmol. Vis. Sci. 46, 2388-2395 (2005).
[86] Sosne, G., Qiu, P., Christopherson, P. L. & Wheater, M. K. Thymosin beta 4 suppression of corneal NFkappaB: A potential anti-inflammatory pathway. Exp. Eye Res. (2007).
[87] Rhaleb, N. E. et al. Long-term effect of N-acetyl-seryl-aspartyl-lysyl-proline on left ventricular collagen deposition in rats with 2-kidney, 1-clip hypertension. Circulation 103, 3136-3141 (2001).
[88] Rasoul, S. et al. Antifibrotic effect of Ac-SDKP and angiotensin-converting enzyme inhibition in hypertension. J. Hypertens. 22, 593-603 (2004).
[89] Peng, H., Carretero, O. A., Liao, T. D., Peterson, E. L. & Rhaleb, N. E. Role of N-acetyl-seryl-aspartyl-lysyl-proline in the antifibrotic and anti-inflammatory effects of the angiotensin-converting enzyme inhibitor captopril in hypertension. Hypertension 49, 695-703 (2007).
[90] Gordon, J. R., Burd, P. R. & Galli, S. J. Mast cells as a source of multifunctional cytokines. Immunol. Today 11, 458-464 (1990).
[91] Frangogiannis, N. G. et al. Resident cardiac mast cells degranulate and release preformed TNF-alpha, initiating the cytokine cascade in experimental canine myocardial ischemia/reperfusion. Circulation 98, 699-710 (1998).
[92] Mori, S., Gibson, G. & McTiernan, C. F. Differential expression of MMPs and TIMPs in moderate and severe heart failure in a transgenic model. J. Card Fail. 12, 314-325 (2006).

1. A population of mammalian post-natal epicardium derived cells (EPDCs), wherein at least 50% of said EPDCs express at least one embryonic gene.

2. A population of isolated post-natal epicardial cells (EPDCs) according to claim 1, wherein at least 50% of said cells express Tbx18 and Raldh2.

3. A population of cells according to claim 2, wherein at least 50% of said cells are capable of expressing at least one of Tie2, PECAM, Flk1 and/or VEGF.

4. A population of cells according to claim 2, wherein at least 50% of said cells are capable of expressing SMαA.

5. A population of cells according to claim 2, wherein at least 50% of said cells are capable of expressing Isl-1, Nkx2.5, and/or Gata4.

6. A population of cells according to claim 2, wherein at least 50% of said cells are capable of expressing procollagen al.

7. A population of isolated post-natal epicardial cells (EPDCs) obtainable by treating epicardial cells with Tβ4, wherein at least 50% of said cells
a) express at least one embryonic gene, preferably Tbx18 and Raldh2; and/or
b) are capable of differentiating into vascular precursor cells, and/or cardiomyocytes, and/or fibroblasts.

8. A population of isolated post-natal epicardial cells characterised in that at least 50% of said cells are capable of differentiating into vascular precursor cells, and/or cardiomyocytes, and/or fibroblasts.

9. A population of cells according to claim 2, wherein at least 50% of said cells express Ki67 and/or phospho-histone H3.

10. A method of obtaining a population of isolated post-natal epicardial cells (EPDCs), comprising the steps of culturing pieces of heart tissue in culture medium comprising about 10-500 ng/ml Tβ4 for sufficient time to permit EPDC outgrowth.

11. The method of claim. 0, wherein the pieces are from 0.5 to 5 mm³.

12. The method of claim 10, wherein the cells are cultured for 12 to 96 hours.

13. The method of claim 10, further comprising the steps of:
a) washing the cells with DPBS, and
b) adding fresh culture medium containing Tβ4.

14. The method of claim 10, wherein the tissue pieces are treated with about 100 ng/ml Tβ4.

15. The population of isolated post-natal epicardial cells (EPDCs) obtained or obtainable by the method of claim 10.

16. A method of promoting EPDC differentiation into endothelial cells comprising culturing the population of cells according to claim 1 in culture medium comprising AcSDKP.

17. A method of promoting EPDC differentiation into cardiomyocytes comprising culturing the population of cells according to claim 1 in culture medium comprising Tβ4.

18. A method of screening for a compound that promotes vascular precursor cell formation, comprising the steps of:
a) exposing a population of cells according to claim 1 to a candidate compound, and
b) comparing vascular precursor cell formation in the presence and absence of the candidate compound.

19. A method of screening for a compound that promotes cardiomyocyte formation, comprising the steps of:
a) exposing a population of cells according to claim 1 to a candidate compound, and
b) comparing cardiomyocyte formation in the presence and absence of the candidate compound.

20. A method of screening for a compound that promotes neovascularisation, comprising the steps of:
a) exposing a population of cells according to claim 1 to a candidate compound, and
b) comparing neovascularisation in the presence and absence of the candidate compound.

21. A transgenic, non-human animal, wherein said animal displays altered Tβ4 expression in the heart.

22. A method of treating or preventing myocardial infarction by administering an effective amount of a population of cells according to claim 1 to a patient in need thereof.

23. A method of treating inflammation in the heart comprising administering an effective amount of Tβ4 to a patient in need thereof.

24. A method of treating or preventing myocardial infarction and/or inflammation in the heart by administering an effective amount of a combination of Tβ4 and a population of cells according to claim 1 to a patient in need thereof.

25. A method of promoting EPDC differentiation into endothelial cells comprising culturing the population of cells according to claim 15 in culture medium comprising AcSDKP.

26. A method of promoting EPDC differentiation into cardiomyocytes comprising culturing the population of cells according to claim 15 in culture medium comprising Tβ4.

27. A method of screening for a compound that promotes vascular precursor cell formation, comprising the steps of:
a) exposing a population of cells according to claim 15 to a candidate compound, and
b) comparing vascular precursor cell formation in the presence and absence of the candidate compound.

28. A method of screening for a compound that promotes cardiomyocyte formation, comprising the steps of:
a) exposing a population of cells according to claim 15 to a candidate compound, and
b) comparing cardiomyocyte formation in the presence and absence of the candidate compound.

29. A method of screening for a compound that promotes neovascularisation, comprising the steps of:
a) exposing a population of cells according to claim 15 to a candidate compound, and
b) comparing neovascularisation in the presence and absence of the candidate compound.

30. A method of treating or preventing myocardial infarction by administering an effective amount of a population of cells according to claim 15 to a patient in need thereof.

31. A method of treating or preventing myocardial infarction and/or inflammation in the heart by administering an effective amount of a combination of Tβ4 and a population of cells according to claim 15 to a patient in need thereof.
